(19) United States
(12) Patent Application Publication
(10) Pub. No.: US 2025/0003980 A1
(43) Pub. Date: Jan. 2, 2025

(54) IDENTIFICATION OF MICROORGANISMS BASED ON IDENTIFICATION OF PEPTIDES USING A LIQUID SEPARATION DEVICE COUPLED WITH A MASS SPECTROMETER AND PROCESSING MEANS

(71) Applicants: Université Claude Bernard Lyon 1, Villeurbanne (FR); Centre National de la Recherche Scientifique (CNRS), Paris (FR); Institut National de la Santé et de la Recherche Médicale (INSERM), Paris (FR); Ecole Normale Superieure de Lyon, Lyon (FR); Hospices Civils de Lyon, Lyon (FR)

(72) Inventors: Jérôme LEMOINE, Lucenay (FR); Maud GREGSON, Lyon (FR); Julie GIL, Venissieux (FR); Romain CARRIERE, Farnay (FR); François VANDENESCH, Rillieux-La-Pape (FR)

(73) Assignees: Université Claude Bernard Lyon 1, Villeurbanne (FR); Centre National de la Recherche Scientifique (CNRS), Paris (FR); Institut National de la Santé et de la Recherche Médicale (INSERM), Paris (FR); Ecole Normale Superieure de Lyon, Lyon (FR); Hospices Civils de Lyon, Lyon (FR)

(21) Appl. No.: 18/578,465

(22) PCT Filed: Jul. 15, 2022
(86) PCT No.: PCT/EP2022/069857

§ 371 Date: Jan. 11, 2024

(30) Foreign Application Priority Data
Jul. 15, 2021 (EP) 21305988.4

(51) Int. Cl. G01N 33/68; G01N 30/02; G01N 30/72; G01N 30/86; G01N 30/88; H01J 49/00

(52) U.S. Cl. CPC G01N 33/6848; G01N 30/7233; G01N 30/8624; G01N 30/88; H01J 49/004; G01N 2030/027; G01N 2030/8831

(57) ABSTRACT

Identification of at least one microorganism present in a sample based on the detection of peptides issued from the cleavage of ribosomal proteins of said microorganism, comprising lysis of microorganism(s) and cleavage of the proteins, obtaining a mixture of peptides, decomplexing the peptide mixture using a liquid separation device coupled with a mass spectrometer, nebulizing the liquid to produce an ion current, receiving the ion current from the ion source, executing on the ion current a series of filtering steps for detecting a transition, receiving data on the transitions, assigning the transitions associated with peptides into two or more contiguous groups, monitoring, and optionally generating a chromatogram or electropherogram, wherein the microorganism is identified according to the detection of said peptide(s).

### FIELD OF THE INVENTION

The present invention relates to a method for identifying microorganisms in a sample, in particular in a biological sample such as a blood sample from a Human being. This method may be used for the detection of septicemia, i.e., blood infections.

### BACKGROUND OF THE INVENTION

The identification of microorganisms responsible for an infection is an essential step in the management of a patient. The rapid access to this identification information is all the more important in the case of symptomatic blood bacteremia induced by the continuous diffusion of pathogens from an infectious site, which can then lead to a sepsis syndrome. From mild then to severe, sepsis can ultimately evolve to a septic shock with which is associated up to 80% mortality.

The rapid identification of the pathogen(s) responsible for the bacteremia is thus a key step to be able to orient the choice of antibiotic therapy, or to proceed to de-escalation of the initial broad spectrum antimicrobial therapy. The expected consequences of such a rapid diagnosis is to reduce the therapeutic side effects in the patient, but also to contain the contribution of antibiotics use to the emergence of new antimicrobial resistant strains or mechanisms.

In other fields than human health, rapid identification of microorganisms present in a sample is also a key factor for adapting the anti-microbial strategy.

Two types of techniques have emerged over the last two decades that have revolutionized microbiological identification, by allowing a singularly shortened identification time compared to previously-used biochemical techniques. These are Maldi-Tof mass spectrometry and molecular biology-based tools.

The principle of pathogen identification by Maldi-Tof (Matrix-assisted laser desorption ionization-Time of flight) mass spectrometry is based on the comparison between an experimental mass spectrum of a fingerprint of low molecular weight proteins released by the lysis of the microbe of interest and a database containing tens of thousands of mass spectra of fingerprints obtained from strains of known microbes. A concordance score is then established to identify the genus of the pathogen or the genus and species of the pathogen. The principle of identification of a microbe by Maldi-Tof was first described in 1999 (Holland et al., 1999) for low molecular weight proteins.

Then the technique was widely deployed in hospitals under the brand names VITEK® MS (Biomerieux) and MALDI Biotyper® (Brucker Daltonics).

When applied on a blood sample, in order to limit the sources of interfering signals on the mass spectrum, blood cells need to be lysed and eliminated. The patent U.S. Pat. No. 8,569,010 discloses a protocol based on the use of sodium dodecyl detergent to support efficient blood cell lysis prior to Maldi-Tof analysis.

Compared to biochemical identification techniques that require prior isolation of the microbe, the Maldi-Tof technique allows to shorten the identification time by about 24 hours and can therefore have a direct impact on mortality statistics and average hospitalization time. In addition, the calculated cost of analysis per sample is very low and the technique leads to very little hospital waste. These advantageous medico-economic characteristics explain the rapid deployment of Maldi-Tof in hospitals.

However, the Maldi-Tof technique has limitations. For example, it is difficult to identify species that are phylogenetically close (i.e. Escherichia coli/Shigella, members of the Citrobacter freundii or Enterobacter cloacae group). Similarly, identification is compromised in the case of polymicrobial infections due to overlapping fingerprints or when one microbe is poorly represented compared to a second predominant species. This situation is typically encountered in situations of bacteremia in the context of poly-microbial infections of digestive origin (peritonitis, intra-abdominal abscess).

This lack of sensitivity is less prominent with the molecular biology-based techniques. Commercial solutions using molecular biology in a broad sense can be distinguished in 4 categories according to whether they employ methods based on i) fluorescence in-situ hybridization (FISH); ii) DNA microarray hybridization; iii) nucleic acid amplification (PCR); or iv) the combination of methods.

The ideal pathogen identification technique should cover the majority of species associated with sepsis, be able to be deployed directly from a positive blood culture aliquot, have the shortest possible turnaround time (ideally less than 1 hour), be economically viable, allow the identification of the different pathogens constituting a polymicrobial infection, and incidentally give an estimate of the relative or even absolute quantification of the pathogen(s). Ideally, this technique should be able to be deployed on a single analysis platform allowing to simultaneously or successively characterize in any way the possible antibiotic resistance mechanism(s) or susceptibility profile associated with the identified pathogen.

Recently, several exploratory studies have evaluated the potential interest of liquid chromatography-mass spectrometry couplings combined with a bottom-up proteomic analysis approach to identify bacteria. In this approach, the protein content of bacteria or yeast is subjected to a specific enzymatic digestion in order to generate peptides, which are then partially separated during the chromatography step before generating mass spectra and/or chromatograms reconstituted on characteristic ions, which will be compared to public or proprietary databases. The mass spectrometry analysis can be conducted in a non-targeted or a targeted manner.

In the case of a non-targeted analysis, the mass spectrometer can operate in such a way as to obtain, for example, information on the exact (monoisotopic) or chemical or molecular or average mass of each of the peptides in the mixture resulting from the enzymatic hydrolysis. In this case, a simple analysis, called MS or MS1, is performed and, as in the case of Maldi-Tof, the experimental fingerprint of all the masses of the peptides (or mass to charge ratio values; m/z) resulting from the enzymatic digestion is compared with all the theoretical fingerprints obtained by the same enzymatic digestion of all the bacterial or yeast proteomes. This is the approach named LC-MS1, as described in (Lasch et al. 2020).

In another implementation, the process comprises in addition to, or as a substitute for peptide mass information, a step wherein peptides are subjected to a fragmentation step. This step can be conditioned by a preliminary observation at a time t of the chromatogram of the n masses of the intact peptides (or values of mass to charge ratio; m/z) which will be then selected one by one to record successively n fragmentation spectra. This mode of operation is called Data Dependent Acquisition (DDA) also known as shotgun proteomics or Information Dependent Acquisition (IDA). The experimental fragmentation spectra of the peptides combined or not with information on their mass are then compared to the theoretical fragmentation spectra of all the peptides resulting from the enzymatic digestion of bacterial or yeast proteomes in order to identify the pathogen(s). This process is used for example in (Boulund et al. 2017)

Alternatively, the peptides are not selected individually from the mass spectrum but systematically fragmented in a blind manner according to the acquisition mode called Data Independent Acquisition (DIA) also known as Sequential Window Acquisition of all Theoretical Mass Spectra (SWATH) or MSE. This is the method used by (Blumenscheit et al., 2020) to detect peptides resulting from the enzymatic digestion of proteins involved in antibiotic resistance.

The targeted acquisition mode is known as Selected Reaction Monitoring (SRM), Multiple Reaction Monitoring (MRM), Parallel Reaction Monitoring (PRM), Multiple Reaction Monitoring-High Resolution (MRM-HR), Multiple Reaction Monitoring cubed (MRM3). Several studies related to the implementation of targeted mass spectrometry for the identification of bacteria or yeasts have been reported, for example to identify bacteria in urine, in tracheobronchial aspirates or from isolated colonies. This method has also been implemented to type bacteria of the genus Acinetobacter, to detect and quantify toxins, to detect antibiotic resistance mechanisms.

The international application WO2011/045544 describes the use of this targeted mass spectrometry method, coupled with a chromatographic separation system, to type strains of Staphylococus aureus from isolated colonies and concomitantly detect virulence factors and n antibiotic resistance. Similarly, the applications WO2012/143535 and WO2012/143534 describe the use of this same method to detect proteins associated with various antibiotic resistance mechanisms.

Nevertheless, it should be noted that up to this day, no diagnostic method for blood infection is based on this method using a peptide separation method combined with their detection by mass spectrometry. The reasons are twofold.

Firstly, the duration of the analysis methods is too long, most often between 30 and 120 minutes of chromatographic separation. This limits the number of samples that can be analyzed per day and increases the cost of an analysis.

Secondly, a targeted mass spectrometry method requires that when a large number of targets are to be sampled, the signals of these targets should be followed only in the chromatographic retention time window during which they are expected to be detected. This ensures that the signal intensity of the compound eluted from the chromatographic separation system will be measured at least 8 times in order to be able to define the shape of the chromatographic peak of the compound with sufficient accuracy. This approach is called “scheduled MRM, scheduled MRM HR, timed MRM, dynamic MRM” according to the mass spectrometry manufacturers. The disadvantage is that if the retention time is unexpectedly changed (e.g. due to the influence of the sample composition or concentration, or wear of the chromatography column), then target compounds may fall outside their scheduled retention window and not be detected. To take this limitation into account, the user usually takes sufficiently wide retention time windows, but this precaution in turn implies a decrease in the number of compounds that can be detected with the method.

The patent EP 3 384 517 describes a technique that overcomes these limitations. The method relies on the monitoring of “sentinel signals” belonging to compounds spread over the chromatographic separation scale. Once a sentinel signal is detected above a defined threshold, then it triggers the monitoring of a set of signals specific of target molecules of interest, until a new sentinel signal is detected. Thus, all target compounds continue to be reliably detected despite any retention time drift.

### SUMMARY OF THE INVENTION

The present patent application describes a method for rapid identification of microorganisms in less than 10 minutes, preferably in 5 to 7 minutes, targeting biomarker peptides selected exclusively from those derived from enzymatic digestion of ribosomal proteins of said microorganisms.

The method is based on a list of peptides that have been thoroughly selected for the implementation of the identification method. These peptides are specific to the species to be identified, and harbor physico-chemical properties that allow them to meet the specifications set for the separation step, i.e., having an optimal peak capacity during a gradient time of about 5 minutes.

The present invention relates to a method for the identification of at least one microorganism present in a sample, based on the detection of peptides issued from the cleavage of ribosomal proteins of said microorganism, comprising the following steps:
a) lysis of microorganism(s) and cleavage of the proteins present in said sample, to obtain a mixture of peptides,
b) decomplexing said peptides mixture using a liquid separation device coupled with a mass spectrometer,
c) nebulizing the liquid eluted from the separation device using an ion source, in order to produce an ion current,
d) receiving said ion current from the ion source using said mass spectrometer and, for each cycle of a plurality of cycles, executing on the ion current a series of filtering steps for detecting a transition, said transition comprising a precursor ion and at least one fragment ion of said precursor ion, said transition being read from a predefined list of transitions using the mass spectrometer, wherein for each transition of the series, the mass spectrometer selects and fragments a precursor ion of the each transition;
e) receiving data concerning a plurality of transitions to be used to monitor the mixture of peptides using the processor,
f) assigning said plurality of transitions into two or more contiguous groups of transitions, into said predefined list of transitions, using the processor,
g) monitoring at least one sentinel transition associated with one sentinel compound in each group of the two or more contiguous groups, wherein said at least one sentinel transition is selected as having the latest expected retention time in the group, using the processor,
h) when the signal of at least one sentinel transition of a group is detected with the mass spectrometer, starting the monitoring of at least one transition in a next contiguous group while stopping the monitoring of the transitions of the preceding group, using the processor,
i) optionally, generating a chromatogram or an electropherogram, from the detection of transitions read from a predefined list with said mass spectrometer, using the processor,
wherein each transition read from the predefined listed is associated to a peptide, and wherein the microorganism is identified according to the detection of said peptide(s).

The present invention also concerns a system for implementing the method as defined above, comprising a mass spectrometer coupled to a liquid separation device, and processing means adapted for the implementation of the steps (e) to (h), in particular adpated:
to receive data concerning a plurality of transitions to be used to monitor the mixture of peptides,
to assign the plurality of transitions into two or more contiguous groups of transitions, into said predefined list of transitions,
to monitor at least one sentinel transition in each group of the two or more contiguous groups,
to start the monitoring of at least one sentinel transition in a next contiguous group, when the signal of at least one sentinel transition of a group is detected by the mass spectrometer, and
optionally, to generate a chromatogram or an electropherogram.

The present invention also relates to a group of peptides adapted for the implementation of the method as described above, wherein said peptides are issued from ribosomal proteins, comprise between 6 and 20 amino acids, and are decomplexed with a mobile phase comprising less than 40% of acetonitrile during the decomplexing step.

### BRIEF DESCRIPTION OF THE FIGURES

FIG. 1 illustrates a chromatogram obtained with the “sentinel-endogenous” method. The intensity of the peptides is shown in function of the retention time. Intensities are expressed in counts par seconds.

Group a: Enterobacterales group, contains transitions associated to peptides common to 18 Enterobacterales and peptides specific to each Enterobacterales (135 transitions)

Group b: Pseudomonas aeruginosa group, contains 47 transitions associated to 12 peptides specific to Pseudomonas aeruginosa (presenting the sequence SEQ ID NO. 290 to 300)

Group c: Staphylococcus aureus_argenteus group, contains 35 transitions associated to 10 peptides specific to Staphylococcus aureus and Staphylococcus argenteus (presenting the sequence SEQ ID NO. 328 to 337), the selected peptides are not present in the other Staphylococcus species of the panel (staphylococcus coagulase negative) (35 transitions)

Group d: Acinetobacter group, contains transitions associated to 16 peptides common to 4 Acinetobacter (presenting the sequences SEQ ID NO. 18 to 33) and peptides specific to each Acinetobacter (128 transitions)

Group e: Enterococcus group, contains transitions associated to peptides common to 2 Enterococcus and peptides specific to each Enterococcus (53 transitions)

Group f: Candida group, contains transitions associated to peptides common to 7 Candida and peptides specific to each Candida (81 transitions)

Group g: Other species group, contains transitions associated to peptides specific to 31 other species (139 transitions)

Group h: “Streptococcus and other” group, contains transitions associated to 17 Streptococcus and specific peptides to certain Streptococcus or group of Streptococcus. The group also contains transitions of 7 other species. It is the only group that is not triggered by sentinel peptides (105 transitions)

FIG. 2 shows the distribution of the following 4 sentinel peptides, issued from trypsin self-digestion, on a chromatographic gradient:

| (SEQ ID NO. 424) | |
|---|---|
| | NKPGVYTK |
| (SEQ ID NO. 425) | |
| | VATVSLPR |
| (SEQ ID NO. 426) | |
| | LGEHNIDVLEGNEQFINAAK |
| (SEQ ID NO. 427) | |
| | IITHPNFNGNTLDNDIMLIK |

FIG. 3: Chromatogram obtained with the identification method of the invention, from a blood culture sample, analyzed with the “endogenous” sentinel peptides: the identified microorganism is Enterococcus faecium.

FIG. 4: Chromatogram obtained with the identification method of the invention, from a blood culture sample, analyzed with the “trypsin” sentinel peptides: the identified microorganism is Enterococcus faecium.

FIG. 5: Chromatogram obtained with the identification method of the invention, from a polymicrobial sample, analyzed with the “trypsin” sentinel peptides: the identified microorganisms are Escherichia coli (SEQ ID NO. 200 and 201) and Streptococcus bovis (SEQ ID NO. 365 and 367).

### DETAILED DESCRIPTION OF EMBODIMENTS OF THE INVENTION

The objective of the method of the invention is to allow the identification of a microorganism in a short time, especially in a time of less than 10 minutes, with an inexpensive process, usable in routine without requiring highly qualified personnel.

In particular, the present invention concerns a method for the identification of at least one microorganism present in a sample, based on the detection of peptides issued from the cleavage of ribosomal proteins of said microorganism, comprising the following steps:
a) lysis of microorganism(s) and cleavage of the proteins present in said sample, to obtain a mixture of peptides,
b) decomplexing said peptides mixture using a liquid separation device coupled with a mass spectrometer,
c) nebulizing the liquid eluted from the separation device using an ion source, in order to produce an ion current,
d) receiving said ion current from the ion source using said mass spectrometer and, for each cycle of a plurality of cycles, executing on the ion current a series of filtering steps for detecting a transition, said transition comprising a precursor ion and at least one fragment ion of said precursor ion, said transition being read from a predefined list of transitions using the mass spectrometer, wherein for each transition of the series, the mass spectrometer selects and fragments a precursor ion of the each transition;
e) receiving data concerning a plurality of transitions to be used to monitor the mixture of peptides using the processor,
f) assigning said plurality of transitions into two or more contiguous groups of transitions, into said predefined list of transitions, using the processor,
g) monitoring at least one sentinel transition associated with one sentinel compound in each group of the two or more contiguous groups, wherein said at least one sentinel transition is selected as having the latest expected retention time in the group, using the processor,
h) when the signal of at least one sentinel transition of a group is detected with the mass spectrometer, starting the monitoring of at least one sentinel transition in a next contiguous group while stopping the monitoring of the transitions of the preceding group, using the processor,
i) optionally, generating a chromatogram or electropherogram, from the detection of transitions read from a predefined list with said mass spectrometer, using the processor,
wherein each transition read from the predefined listed is associated to a peptide, and wherein the microorganism is identified according to said peptide(s) that are detected.

Each of the steps of this identification method is presented with more details hereafter.

In the sense of the invention, the term “microorganism” designates a bacteria or a yeast.

In a specific embodiment of the invention, the microorganism is a pathogenic microorganism causing diseases to human beings. In particular, the microorganism is chosen among the most prevalent microorganisms causing bacterial infection, sepsis, or urinary tract infections.

The microorganisms that can be identified by the method of the invention can be a group of microorganisms representing a family, a genus or a species of microorganisms, pathogenic or not.

For example, the following bacteria belonging to the ESKAPE group are examples of identified microorganisms:
Enterococcus faecium,
Staphylococcus aureus,
Klebsiella pneumoniae,
Acinetobacter baumannii,
Pseudomonas aeruginosa, and
Enterobacter spp.

As other examples of microorganisms that can be identified, there may be mentioned:
Yeasts, for example Candida krusei,
Streptococcus, for example Streptococcus pneumoniae,
Coagulase negative Staphylococcus.

In the sense of the invention, the term “sample” designates:
a biological sample obtained from a mammal, chosen among the group consisting of: blood, serum, lymph, mucus, stink, saliva, tracheobronchial aspirate, cerebrospinal fluid and urine, or
a sample chosen among the group consisting of: used waters, food, drink, soil sample and surface sample.

The sample may comprise one or multiple microorganisms. The method is adapted for the identification of at least one microorganism, and therefore can be implemented for identifying multiple microorganisms.

Advantageously, the method of the invention is realized in a short time, in any case in less than 10 minutes. Accordingly, this method is adapted for diagnosis and in particular for the diagnosis of sepsis in human beings.

In a preferred embodiment, the sample is a biological sample obtained from a human being, chosen among the group consisting of: blood, serum, lymph, mucus, stink, saliva, tracheobronchial aspirate, cerebrospinal fluid and urine, and is in particular a blood sample.

### Step (a) of the Method

Before any step of analysis, microorganisms present in the sample are preferentially pelleted with centrifugation, filtration, acoustophoresis, levitation or spinning.

In a specific embodiment of the invention, step (a) comprises a preliminary substep of elimination of peptides that are not issued from the cleavage of ribosomal proteins. This is achieved, in particular, by addition of a surfactant into the assayed sample.

Lysis of microorganism(s) present in the sample and cleavage of the proteins are performed to obtain a mixture of peptides. In a specific embodiment, both actions are performed concomitantly.

Since the characterization of the microorganisms comes from proteins, it is necessary to process the sample before analysis by mass spectrometry. To generate peptides from the proteins present in the sample, it is possible to digest these proteins with a proteolytic enzyme (protease), for example trypsin or pepsin, or by the action of a chemical reagent, for example treatment with bromide cyanogen (CNBr) or treatment with hydroxyl radicals (H₂O₂).

Cleavage of proteins by enzymatic digestion is however preferable because it is easier to control and less denaturing for the structure of proteins compared to treatment with chemical reagent and particularly specific.

Enzymatic digestion is the action of one (or more) enzyme(s) which, under certain reaction conditions, will allow production of peptides from a protein. Enzymes that cut proteins in specific places, thus carrying out proteolysis are called proteases. Each protease usually has a specific cleavage site among an amino acid sequence that they are able to recognize.

The international application WO2005/098071 describes proteases that can be cited as examples:
Pepsin that hydrolyzes peptide bonds level with the amine function of aromatic amino acids (Tyr, Trp, Phe). It is used at acidic pH.
Endolysin that cuts the peptide bond of the CO group of lysines.
Trypsin that cuts the peptide bond at the level of the carboxylic group of the Lys and Arg residues.

In the method of the invention, cleavage of the proteins is preferably performed by digestion with the trypsin enzyme.

Advantageously the temperature of incubation during the step (a) of lysis and cleavage of proteins is of about 37° C.

The generation of the peptide mixture can be performed by simple dissolution. It can also be sped up using various ancillary processes such as pressurization, a microwave oven or even an ultrasound device. Lysis of the cells present in the sample may thus be more efficient with the use of one of the three methods.

### Step (b)

The decomplexion of the peptides designates a partial step of separation of the peptide. This step is performed by a liquid separation technology such as liquid chromatography or capillary electrophoresis.

Liquid chromatography (LC) is a separation technique in which the mobile phase is a liquid. It can be carried out either in a column or a plane. It includes in particular high-performance liquid chromatography (HPLC), normal phase liquid chromatography (NPLC) and reversed phase liquid chromatography (RPLC).

Capillary electrophoresis (CE) is a family of separation methods performed in submillimeter diameter capillaries and in micro-and nanofluidic channels. It includes in particular capillary zone electrophoresis (CZE), capillary gel electrophoresis (CGE), capillary isoelectric focusing (CIEF), capillary isotachophoresis and micellar electrokinetic chromatography (MEKC).

In a preferred embodiment of the invention, the decomplexing step is performed by reverse phase liquid chromatography.

In an embodiment of the invention, the step of decomplexing the peptides mixture is carried out with a mobile phase comprising less than 40% of acetonitrile, on a reverse phase column. Peptides are selected as a function of their sequence length, as this feature is correlated with the retention factor k, hence the percentage of acetonitrile required for their elution out of the reverse phase column.

Acetonitrile (methyl cyanide) is a polar aprotic solvent.

### Step (c)

The method is performed on a mass spectrometer that is coupled to the liquid separation device. Analysis with the mass spectrometry analysis is conducted in a targeted manner.

Typically, mass spectrometry (MS) is an analytical method where the liquid to be analyzed is ionized, using an ion source, in order to produce an ion current. The “nebulizing” of the liquid containing the mixture of peptides is well known by the person skilled in the art.

In a preferred embodiment, the mass spectrometry is of the type tandem mass spectrometry MS/MS, preferentially a parallel reaction monitoring (PRM) or a multiple reaction monitoring MRM.

In this embodiment, transitions are MRM transitions.

### Step (d)

The mass spectrometer receives said ion current from the ion source and, for each cycle of a plurality of cycles, executes on the ion current a series of filtering steps for detecting a transition, said transition comprising a precursor ion and at least one fragment ion of said precursor ion, said transition being read from a predefined list of transitions using the mass spectrometer, wherein for each transition of the series, the mass spectrometer selects and fragments a precursor ion of the each transition.

In the sense of the invention, a “predefined list of transitions” designates a finite list of transitions, i.e., of specific pairs of m/z values associated to a precursor and fragment ions, wherein each transition is associated to a specific peptide. In other words, the mass spectrometer systematically monitors these transitions that are each associated to a specific peptide, and that have been defined before the analysis.

This process does not analyze in real-time the precursor ion spectrum, and does not add any information to the list. Such real-time analysis has been described, for example, in the international application WO 2014/116711.

The predefined list of transitions has been established on the basis of the table 1 regrouping 423 peptides presented below, having the sequences SEQ ID NO. 1 to SEQ ID NO. 423.

This predefined list contains transitions that are each associated to a specific peptide, possibly present in the mixture of peptides that is analyzed, and therefore that is monitored.

In a specific embodiment of the invention, the predefined list comprises at least one transition that is associated to a peptide presenting a peptide sequence selected from SEQ ID NO. 1 to SEQ ID NO. 423.

Advantageously, the predefined list comprises transitions associated to at least two, three, four, five, six, seven, eight, nine ten, twenty, thirty, forty, fifty, sixty, seventy, eighty, ninety, hundred, two hundred, three hundred or four hundred distinct peptides chosen among the group consisting of peptides having a peptide sequence selected from SEQ ID NO. 1 to SEQ ID NO. 423.

In particular, the predefined list comprises transitions associated to the 423 peptides as listed in table 1.

In another embodiment, the predefined list comprises transitions associated exclusively to at least one of the following group of peptides:
peptides specific to Enterobacterales, in particular presenting a sequence chosen among SEQ ID NO. 155 to 158, SEQ ID NO. 168 to 180, SEQ ID NO. 200 to 201, SEQ ID NO. 223 to 229, SEQ ID NO. 245 to 264, SEQ ID NO. 274 to 277, SEQ ID NO. 282 to 289, SEQ ID NO. 306 to 307, SEQ ID NO. 320 to 327, SEQ ID NO. 393 to 395, SEQ ID NO. 398 to 409 and SEQ ID NO. 422 to 423;
peptides specific to Acinetobacter, in particular presenting a sequence chosen among SEQ ID NO. 13 to SEQ ID NO. 42;
peptides specific to Enterococcus, in particular presenting a sequence chosen among SEQ ID NO. 181 to SEQ ID NO. 199;
peptides specific to Candida, in particular presenting a sequence chosen among SEQ ID NO. 81 to SEQ ID NO. 150;
peptides specific to Staphylococcus, in particular presenting a sequence chosen among SEQ ID NO. 328 to SEQ ID NO. 345 and SEQ ID NO. 410 to SEQ ID NO. 421;
peptides specific to Streptococcus, in particular presenting a sequence chosen among SEQ ID NO. 349 to SEQ ID NO. 392;
peptides specific to Pseudomonas aeruginosa, in particular presenting a sequence chosen among SEQ ID NO. 290 to SEQ ID NO. 300; or
peptides specific to other genus/species, in particular presenting a sequence chosen among SEQ ID NO. 1 to SEQ ID NO. 12, SEQ ID NO. 43 to 80, SEQ ID NO. 151 to 154, SEQ ID NO. 159 to 167, SEQ ID NO. 202 to 222, SEQ ID NO. 230 to 244, SEQ ID NO. 265 to 273, SEQ ID NO. 278 to 281, SEQ ID NO. 301 to 305, SEQ ID NO. 308 to 319, SEQ ID NO. 346 to 348, and SEQ ID NO. 396 to 397.

### Step (e)

The plurality of generated transitions are data that are received by a processor and can be used to monitor the mixture of peptides, using said processor.

The term “processor” means, in the sense of the invention, any digital circuit which performs operations on some external data source. In particular, the processor is a computer. In the present case, the external data source is the mass spectrometer, and the transmitted data are the plurality of transitions.

In a classical way, the computer is adapted to execute code instructions to implement part of the data processing. It may also include a data storage module (a memory, for example flash) and advantageously a user interface (typically a screen), and biometric acquisition means.

### Steps (f), (g), (h)

Said plurality of transitions is assigned into two or more contiguous groups of transitions, into said predefined list of transitions, using the processor.

According to an embodiment, the two or more contiguous groups of transitions are associated with groups of peptides, each of the peptides being specific of a microorganism genus and/or species.

Then a step of monitoring at least one sentinel transition associated with one sentinel compound in each group of the two or more contiguous groups is performed, wherein said at least one sentinel transition is selected as having the latest expected retention time in the group, using the processor.

Sentinel compounds are presented in more details in a dedicated chapter.

At the next step, when the signal of at least one sentinel transition of a group is detected with the mass spectrometer, the monitoring of at least one sentinel transition in a next contiguous group starts, while the monitoring of the transitions of the preceding group is stopped, using the processor.

### Optional Step (i)

A chromatogram or electropherogram may be generated, from the detection of transitions read from a predefined list with said mass spectrometer, using the processor.

In this chromatogram or electropherogram, each peptide is represented by a peak that is “reconstituted” from the data obtained with the mass spectrometer.

While this step is not mandatory, it is useful for visual interpretation of the results.

### Detected Peptides

The method for identification of microorganism(s) is based on the detection of peptides issued from the cleavage of ribosomal proteins belonging to said microorganism. Ribosomal proteins are known to be abundant, stable over time, that is to say not prone to mutations. These proteins are part of the ribosome and, among other things, translate genes encoded on messenger RNAs. There are two types of ribosomal proteins depending on the ribosome subunit to which it belongs. The letter L (for large) qualifies proteins for the large subunit and the letter S (for small) for the small subunit.

In the method of the invention, each transition read from the predefined list is associated to a peptide that is further represented by a peak on the edited chromatogram or electropherogram.

As shown in the examples section, based on the peaks present in said chromatogram or electropherogram, the microorganism can be identified.

In an embodiment of the invention, detection of one peptide specific of the genus and/or species of a microorganism is sufficient to identify such microorganism.

In another embodiment of the invention, detection of at least two peptides, specific of the genus and/or species of a microorganism, is used to identify said microorganism.

Advantageously, in the process of the invention, two or more distinct species can be identified with the detection of two or more peptides present in a same sample, each one being specific of a genus and/or a species.

Each transition is associated to a peptide issued from a ribosomal protein from a microorganism, hereafter designated as a “biomarker peptide”, that has been thoroughly selected according to the features presented below.

First, these biomarker peptides need to be specific to the genus and/or species of the microorganisms to be identified.

Secondly, the biomarker peptides need to harbor physico-chemical properties that allow them to meet the specifications set for the liquid separation, i.e., an optimal peak capacity during a short gradient time of few minutes.

Peak capacity is defined by the following equation:

$$P = 1 + \frac{\sqrt{N}}{4} \times \frac{B Δ c}{B Δ c ( \frac{t 0}{tg} ) + 1}$$

where:
N is the column efficiency calculated in isocratic mode;
B is the slope of the linear dependency of In k versus the percentage of the organic solvent in the mobile phase (B=2.303*0.25 \sqrt{Molecular} weight);.
Δc is the difference of the composition of the mobile phase during the gradient time (tg), with t0 corresponding to the void retention time.

For example, in an experimental set-up employing a column of 100 mm length, an internal diameter of 1 mm, a particle size of 3.5 μm, a flow of 100 μL/min and a reduced gradient time (4.12 min) to implement a rapid turnaround time, the peak capacity reaches an optimal value as soon as 30-35% of acetonitrile in the gradient solvent. Exceeding this percentage implies that more peptides will be eluted at the same peak capacity, which increases the probability of interference in the signals associated with the targets of interest.

Thirdly, the biomarker peptides are also selected as a function of their sequence length, as this feature is correlated with the retention factor k, hence the percentage of acetonitrile required for their elution out of the reverse phase column, in particular for elution out of an octadecyl reverse phase column. Among all the peptide candidates identified as specific biomarkers of the species, only those containing between 6 and 20 amino-acids were thus finally kept in the identification assay to ensure no more than 40% of acetonitrile in the gradient solvent.

In a preferred embodiment of the invention, the predefined list comprises at least one transition that is associated to a peptide comprising between 6 and 20 amino-acids, and that is decomplexed during step (b) with a mobile phase comprising less than 40% of acetonitrile. More specifically, the predefined list comprises at least one transition that is associated to a peptide presenting a peptide sequence selected from SEQ ID NO. 1 to SEQ ID NO. 423, as presented in table 1 below.

Advantageously, each transition of the predefined list is associated to a peptide selected among the group of peptides comprising, or consisting of, peptides having the sequences as shown in SEQ ID NO. 1 to SEQ ID NO. 423.

In a specific implementation of the process, in the predefined list, at least one transition is associated with at least one peptide presenting a sequence selected from SEQ ID NO. 1 to SEQ ID NO. 423.

In another embodiment, the predefined list comprises transitions associated exclusively to at least one of the following group of peptides:
peptides specific to Enterobacterales, in particular presenting a sequence chosen among SEQ ID NO. 155 to 158, SEQ ID NO. 168 to 180, SEQ ID NO. 200 to 201, SEQ ID NO. 223 to 229, SEQ ID NO. 245 to 264, SEQ ID NO. 274 to 277, SEQ ID NO. 282 to 289, SEQ ID NO. 306 to 307, SEQ ID NO. 320 to 327, SEQ ID NO. 393 to 395, SEQ ID NO. 398 to 409 and SEQ ID NO. 422 to 423;
peptides specific to Acinetobacter, in particular presenting a sequence chosen among SEQ ID NO. 13 to SEQ ID NO. 42;
peptides specific to Enterococcus, in particular presenting a sequence chosen among SEQ ID NO. 181 to SEQ ID NO. 199;
peptides specific to Candida, in particular presenting a sequence chosen among SEQ ID NO. 81 to SEQ ID NO. 150;
peptides specific to Staphylococcus, in particular presenting a sequence chosen among SEQ ID NO. 328 to SEQ ID NO. 345 and SEQ ID NO. 410 to SEQ ID NO. 421;
peptides specific to Streptococcus, in particular presenting a sequence chosen among SEQ ID NO. 349 to SEQ ID NO. 392;
peptides specific to Pseudomonas aeruginosa, in particular presenting a sequence chosen among SEQ ID NO. 290 to SEQ ID NO. 300; or
peptides specific to other genus/species, in particular presenting a sequence chosen among SEQ ID NO. 1 to SEQ ID NO. 12, SEQ ID NO. 43 to 80, SEQ ID NO. 151 to 154, SEQ ID NO. 159 to 167, SEQ ID NO. 202 to 222, SEQ ID NO. 230 to 244, SEQ ID NO. 265 to 273, SEQ ID NO. 278 to 281, SEQ ID NO. 301 to 305, SEQ ID NO. 308 to 319, SEQ ID NO. 346 to 348 and SEQ ID NO. 396 to 397.

TABLE 1
Peptides specific of microorganism genus and/or species

| SEQ | | | | Uniprot ID |
|---|---|---|---|---|
| ID | | | | (Accession |
| NO. | Species | Peptide sequence | Protein | number) |
| 1 | Abiotrophia defectiva | SDEEAHALLK | 50S-L5 | A0A1F1LMP7 |
| 2 | Abiotrophia defectiva | GAMVLPHGTGK | 50S-L1 | A0A1F1LHQ2 |
| 3 | Abiotrophia defectiva | GSEAVSLTVNR | Ribosomal | W1Q6D2 |
| | | | L25p | |
| | | | family | |
| | | | protein | |
| 4 | Abiotrophia defectiva | FVDQMITLGK | 50S-L17 | W1Q318 |
| 5 | Abiotrophia defectiva | AAALANLVEGSIVEGTVAR | 30S-S1 | A0A1F1LR69 |
| 6 | Abiotrophia defectiva | EYAVVNLEALNR | 50S-L15 | W1Q3H8 |
| 7 | Abiotrophia defectiva | LGFEGGQTQLFR | 50S-L15 | W1Q3H8 |
| 8 | Abiotrophia defectiva | ELDLIGVGYR | 50S-L6 | W1Q666 |
| 9 | Achromobacter | LMQVILAPIVTEK | 50S-L23 | A0A427X0D6 |
| | xylosoxidans and | | | |
| | Achromobacter | | | |
| | denitrificans | | | |
| 10 | Achromobacter | VVGALGQILGPR | 50S-L1 | A0A427X058 |
| | xylosoxidans and | | | |
| | Achromobacter | | | |
| | denitrificans | | | |
| 11 | Achromobacter | VIEPLITLGK | 50S-L17 | A0A3R9MTD8 |
| | xylosoxidans and | | | |
| | Achromobacter | | | |
| | denitrificans | | | |
| 12 | Achromobacter | GNTGETLIQLLESR | 30S-S4 | A0A427X0H0 |
| | xylosoxidans and | | | |
| | Achromobacter | | | |
| | denitrificans | | | |
| 13 | Acinetobacter baumannii | ILYEIEGVNEDLAR | 50S-L16 | V5VB35 |
| 14 | Acinetobacter baumannii | TDLPEFAPGDTVVVQVK | 50S-L19 | A0A5R9HP13 |
| 15 | Acinetobacter baumannii | ATIANVNASDEER | 30S-S14 | A0A3S8VGD7 |
| 16 | Acinetobacter baumannii | SGTTGNIEAATK | 50S-L18 | V5V9P3 |
| 17 | Acinetobacter baumannii | STGESVAVAK | 50S-L24 | V5V9N7 |
| 18 | Acinetobacter common | TLEQYFGR | 30S-S9 | A0A429H485 |
| 19 | Acinetobacter common | QGLGIAIVSTSK | 30S-S8 | A0A3G9FV19 |
| 20 | Acinetobacter common | GGFTVDIGPVR | 30S-S1 | A0A4Y3J2V9 |
| 21 | Acinetobacter common | GIQPVSPWGQK | 50S-L2 | A0A3R9R8G4 |
| 22 | Acinetobacter common | VEGDIVSLETLK | 50S-L15 | A0A0B2XTW3 |
| 23 | Acinetobacter common | AGDAAPMAYVELVDR | 50S-L17 | A0A429H5X2 |
| 24 | Acinetobacter common | AALDYGLK | 30S-S11 | A0A429H627 |
| 25 | Acinetobacter common | EISMNIK | 30S-S13 | A0A2K8UNJ7 |
| 26 | Acinetobacter common | EPDLTGADLDAR | 50S-L11 | A0A0M3BX59 |
| 27 | Acinetobacter common | NVMEIPR | 50S-L5 | A0A0M3BYW4 |
| 28 | Acinetobacter common | LADEVEATLK | 30S-S1 | A0A0B2XY32 |
| 29 | Acinetobacter common | FNVLTSPHVNK | 30S-S10 | A0A0M3BZ77 |
| 30 | Acinetobacter common | VNIASIQVK | 30S-S4 | A0A2N6VEF0 |
| 31 | Acinetobacter common | LIDIVQPTDK | 30S-S10 | A0A2K8UNK9 |
| 32 | Acinetobacter common | AFTVQGVALTK | 50S-L15 | A0A0B2XTW3 |
| 33 | Acinetobacter common | IFEDGEIVTGVISGK | 30S-S1 | A0A4Y3J2V9 |
| 34 | Acinetobacter lwoffii and | GMAMNPVDHPHGGGEGR | 50S-L2 | A0A2N6VEC5 |
| | Acinetobacter ursingii | | | |
| 35 | Acinetobacter lwoffii and | AVEQLFGVEVVK | 50S-L23 | A0A2K8UNH0 |
| | Acinetobacter ursingii | | | |
| 36 | Acinetobacter lwoffii and | QLGEDPWLAIMNR | 30S-S1 | A0A4Y3J2V9 |
| | Acinetobacter ursingii | | | |
| 37 | Acinetobacter lwoffii and | SIAESIVYGALDR | 30S-S7 | A0A2K8UKR0 |
| | Acinetobacter ursingii | | | |
| 38 | Acinetobacter pittii | LQLAPVK | 50S-L6 | A0A0M3C360 |
| 39 | Acinetobacter pittii | ILYEIEGVNEELAR | 50S-L16 | A0A0M3BYW7 |
| 40 | Acinetobacter pittii | LFEDFAK | 50S-L10 | A0A429KCI7 |
| 41 | Acinetobacter pittii | AQVLGDTVGVQVFK | 50S-L23 | A0A3G6YJ34 |
| 42 | Acinetobacter pittii | QPLELLEVTEK | 30S-S9 | A0A0M3BW35 |
| 43 | Actinomyces | GTHFHPGDGVGR | 50S-L27 | A0A0V8RR43 |
| | odontolyticus | | | |
| 44 | Actinomyces | IQVFQGVVIAR | 50S-L19 | A0A0V8RTA2 |
| | odontolyticus | | | |
| 45 | Actinomyces | TAGLTGENLVELLEMR | 30S-S4 | A0A21112R6 |
| | odontolyticus | | | |
| 46 | Actinomyces | VEDGIEGLVHISELAQR | 30S-S1 | A0A0V8RR48 |
| | odontolyticus | | | |
| 47 | Aerococcus viridans | EATASAVSAQR | 50S-L9 | A0A2J9PM59 |
| 48 | Aerococcus viridans | GASSGWGK | 50S-L15 | A0A2N6UGG4 |
| 49 | Aerococcus viridans | MLDQAASK | 30S-S20 | A0A2N6UFJ2 |
| 50 | Aerococcus viridans | IAIQEAHK | 30S-S2 | A0A2X0UMZ9 |
| 51 | Aerococcus viridans | VGDTLELVVIK | 30S-S1 | A0A2N6UEX7 |
| 52 | Aerococcus viridans | YALSEAIELLK | 50S-L1 | A0A2J9PLY4 |
| 53 | Aerococcus viridans | NWVVLDATDVPLGR | 50S-L13 | A0A2J9PLK4 |
| 54 | Bacillus simplex and | VATIEYDPNR | 50S-L2 | A0A2A8UL96 |
| | Bacillus cereus | | | |
| 55 | Bacillus simplex and | MYAIIETGGK | 50S-L21 | A0A2B11YY8 |
| | Bacillus cereus | | | |
| 56 | Bacillus simplex and | LDLPSGVDIEIK | 30S-S10 | A0A0G8F725 |
| | Bacillus cereus | | | |
| 57 | Bacillus simplex and | WLGGTLTNFETIQK | 30S-S2 | A0A270AZC9 |
| | Bacillus cereus | | | |
| 58 | Bacillus simplex and | EQLIFPEIDYDK | 50S-L5 | A0A2A8RT89 |
| | Bacillus cereus | | | |
| 59 | Bacillus simplex and | MADAILEAK | 30S-S2 | A0A2B0MPM |
| | Bacillus cereus | | | 4 |
| 60 | Bacillus simplex and | TGTVTFDVTK | 50S-L1 | A0A2B1K594 |
| | Bacillus cereus | | | |
| 61 | Bacteroides fragilis | GITGEVLLQMLEGR | 30S-S4 | A0A4P8L9Z0 |
| 62 | Bacteroides fragilis | LLVVLPEANK | 50S-L4 | A0A4P8L976 |
| 63 | Bacteroides fragilis | QLTPHPWDALDPNLQVGDK | 30S-S1 | A0A081TN02 |
| 64 | Bacteroides fragilis and | AFAEQLVNLTVK | 50S-L7/ | A0A081TQY3 |
| | Bacteroides | | 12 | |
| | thetaiotamicron and | | | |
| | Bacteroides vulgatus | | | |
| 65 | Bacteroides fragilis and | LNVVILDFDDEK | 30S-S1 | A0A3E5GBY9 |
| | Bacteroides | | | |
| | thetaiotamicron and | | | |
| | Bacteroides vulgatus | | | |
| 66 | Bacteroides fragilis and | VINGLGIAIISTSK | 30S-S8 | A0A0P0LFV1 |
| | Bacteroides | | | |
| | thetaiotamicron and | | | |
| | Bacteroides vulgatus | | | |
| 67 | Bacteroides | VGEMIAK | 50S-L18 | A0A0P0F082 |
| | thetaiotaomicron | | | |
| 68 | Bacteroides | FIPVYVTENMVGHK | 30S-19 | A0A0P0LM37 |
| | thetaiotaomicron and | | | |
| | Bacteroides vulgatus | | | |
| 69 | Bacteroides | GAPEGFVAPVTPGR | 50S-L16 | A0A0P0EVD2 |
| | thetaiotaomicron and | | | |
| | Bacteroides vulgatus | | | |
| 70 | Bacteroides | TSFDVVLK | 50S-L7/ | A0A0P0F576 |
| | thetaiotaomicron and | | L12 | |
| | Bacteroides vulgatus | | | |
| 71 | Bacteroides vulgatus | AGDTITVAYR | 50S-L19 | A0A173YPU1 |
| 72 | Bacteroides vulgatus | ALYNVIPER | 50S-L25 | A0A0P0M4P9 |
| 73 | Campylobacter coli and | HSGYFGSVK | 50S-L13 | A0A5L4WW85 |
| | Campylobacter fetus and | | | |
| | Capylobacter jejuni | | | |
| 74 | Campylobacter coli and | LDVGDALLVR | 50S-L25 | A0A317XDX5 |
| | Campylobacter fetus and | | | |
| | Capylobacter jejuni | | | |
| 75 | Campylobacter coli and | SLGSNNSANVVR | 30S-S5 | A0A0Q2L2L4 |
| | Campylobacter fetus and | | | |
| | Capylobacter jejuni | | | |
| 76 | Campylobacter coli and | FMYGVSEK | 30S-S4 | A0A3Z9F5H4 |
| | Campylobacter fetus and | | | |
| | Capylobacter jejuni | | | |
| 77 | Campylobacter coli and | LAAELLDAANSK | 30S-S7 | A0A1T1Z147 |
| | Campylobacter fetus and | | | |
| | Capylobacter jejuni | | | |
| 78 | Campylobacter coli and | ALMDLGSFR | 30S-S13 | A0A5L4WVV9 |
| | Campylobacter fetus and | | | |
| | Capylobacter jejuni | | | |
| 79 | Campylobacter coli and | MLDIVAATPDTVDSLTK | 30S-S10 | A0A5L4WXB7 |
| | Campylobacter fetus and | | | |
| | Capylobacter jejuni | | | |
| 80 | Campylobacter coli and | LLELIGVPFTK | 50S-L5 | A0A400M8E2 |
| | Campylobacter fetus and | | | |
| | Capylobacter jejuni | | | |
| 81 | Candida albicans (yeast) | VETGNFSWGSEGVSR | 40S-S8 | Q59T44 |
| 82 | Candida albicans (yeast) | IIVAPIATETAMK | 60S-L25 | C4YSV1 |
| 83 | Candida albicans (yeast) | YGNVNNDFVLLK | 60S-L3 | C4YKL4 |
| 84 | Candida albicans (yeast) | QVVFEIPGESH | 40S-S7 | Q5AJ93 |
| 85 | Candida albicans (yeast) | SVDAALLSEIK | 60S-S6 | Q9P834 |
| 86 | Candida albicans (yeast) | AVASGASVVSK | 60S-L24 | C4YGY5 |
| 87 | Candida auris (yeast) | APSTFER | 40S-S1 | A0A2H0ZC96 |
| 88 | Candida auris (yeast) | AVEVPEK | 60S-L13 | A0A2H0ZMU1 |
| 89 | Candida auris (yeast) | TAYETLR | 60S-L13 | A0A2H0ZMU1 |
| 90 | Candida auris (yeast) | LVMVTGGK | 40S-S4 | A0A2H0ZMW6 |
| 91 | Candida auris (yeast) | VGVLPEDK | 40S | A0A2H1A2S6 |
| | | | ribosomal | |
| | | | protein | |
| | | | S9-A | |
| 92 | Candida auris (yeast) | AVDPFAK | 40S-S1 | A0A2H0ZC96 |
| 93 | Candida auris (yeast) | EVGLGFK | 40S | A0A2H0ZRL1 |
| | | | ribosomal | |
| | | | protein | |
| | | | S11-A | |
| 94 | Candida auris (yeast) | VAPLPLAAK | 60S-L8 | A0A2H0ZJ33 |
| 95 | Candida auris (yeast) | LLAGLPIR | 40S-S3 | A0A2H0ZW29 |
| 96 | Candida auris (yeast) | SPLDVFSEEAK | 40S-S2 | A0A2H0ZCD6 |
| 97 | Candida auris (yeast) | SIVSEVSGLAPYER | 60S-L36 | A0A2H1A421 |
| 98 | Candida auris (yeast) | TINPLGGFVR | 60S-L3 | A0A2H0ZM86 |
| 99 | Candida auris (yeast) | VIDLQAPAQIVK | 40S-S20 | A0A2H1A705 |
| 100 | Candida auris (yeast) | GQLPQVPIIVK | 60S-L28 | A0A510P2R6 |
| 101 | Candida auris (yeast) | ALAIFVPVPSLVGYR | 40S-S7 | A0A2H1A787 |
| 102 | Candida common | TSFFQALGVPTK | 60S | C4YTG6 |
| | | | acidic | |
| | | | ribosomal | |
| | | | protein | |
| | | | P0 | |
| 103 | Candida common | AFLIEEQK | 60S | A0A0W0EQF1 |
| | | | ribosomal | |
| | | | protein | |
| | | | L34-B | |
| 104 | Candida common | LLGTAFK | 40S-S23 | A0A2H0ZN64 |
| 105 | Candida common | IGPLGLSPK | 60S-L12 | C4YPY4 |
| 106 | Candida common | FQTPAEK | 60S-L3 | C4YKL4 |
| 107 | Candida common | TFGASVR | 60S | W0T8U8 |
| | | | ribosomal | |
| | | | protein | |
| | | | L33-B | |
| 108 | Candida glabrata (yeast) | ALSEQAEAR | 60S | A0A0W0CHG0 |
| | | | ribosomal | |
| | | | protein | |
| | | | L19-B | |
| 109 | Candida glabrata (yeast) | TLVQAPR | 40S-S27 | A0A0W0CD45 |
| 110 | Candida glabrata (yeast) | TPVTLAR | 40S-S28 | A0A0W0CZC9 |
| 111 | Candida glabrata (yeast) | DDEVLVTR | 60S | A0A0W0D7E5 |
| | | | ribosomal | |
| | | | protein | |
| | | | L26-B | |
| 112 | Candida glabrata (yeast) | LAASVIGAGK | 60S | A0A0W0CHG0 |
| | | | ribosomal | |
| | | | protein | |
| | | | L19-B | |
| 113 | Candida glabrata (yeast) | VISDILTR | 40S-S1 | A0A0W0D935 |
| 114 | Candida glabrata (yeast) | QFLELTR | 60S-L38 | A0A0W0CD94 |
| 115 | Candida glabrata (yeast) | GVIGVIAGGGR | 60S-L2 | A0A0W0D2G7 |
| 116 | Candida glabrata (yeast) | YTLDVESFK | 60S-L27 | A0A0W0C5M3 |
| 117 | Candida glabrata (yeast) | LLEMSTEDFIK | 40S-S15 | A0A0W0CBJ6 |
| 118 | Candida glabrata (yeast) | GFSLAEIK | 60S-L13 | A0A0W0D4Y9 |
| 119 | Candida glabrata (yeast) | QIVFEIPETH | 40S-S7 | A0A0W0C9H1 |
| 120 | Candida kefyr | QYATVSR | 60S | W0TG39 |
| | (Kluyveromyces | | ribosomal | |
| | marxianus) (yeast) | | protein | |
| | | | L34-B | |
| 121 | Candida kefyr | TPGGVLR | 60S | W0TG39 |
| | (Kluyveromyces | | ribosomal | |
| | marxianus) (yeast) | | protein | |
| | | | L34-B | |
| 122 | Candida kefyr | VIEQPITSETAMK | Ribosomal | W0T9W9 |
| | (Kluyveromyces | | protein | |
| | marxianus) (yeast) | | L23 | |
| 123 | Candida kefyr | VVYALTTIR | 40S-S18 | W0TCR0 |
| | (Kluyveromyces | | | |
| | marxianus) (yeast) | | | |
| 124 | Candida kefyr | AVVGASLELIK | 60S-L24 | W0T4H8 |
| | (Kluyveromyces | | | |
| | marxianus) (yeast) | | | |
| 125 | Candida kefyr | LWTLVPEEK | Ribo- | W0T7U8 |
| | (Kluyveromyces | | somal_L18e | |
| | marxianus) (yeast) | | super | |
| | | | family | |
| 126 | Candida kefyr | EGDILVLMESER | 40S-S28 | P33286 |
| | (Kluyveromyces | | | |
| | marxianus) (yeast) | | | |
| 127 | Candida krusei (Pichia | AVVVPEQTAYR | 60S-L13 | A0A099P3K9 |
| | kudriavzevii) (yeast) | | | |
| 128 | Candida krusei (Pichia | ALEQVNLK | 60S-L3 | A0A099NWB6 |
| | kudriavzevii) (yeast) | | | |
| 129 | Candida krusei (Pichia | VIQSPITSESATK | 60S-L25 | A0A099P8P7 |
| | kudriavzevii) (yeast) | | | |
| 130 | Candida krusei (Pichia | VFLDVGLQR | 60S-L5 | A0A099NWE2 |
| | kudriavzevii) (yeast) | | | |
| 131 | Candida krusei (Pichia | TITPMGGFVR | 60S-L3 | A0A099NWB6 |
| | kudriavzevii) (yeast) | | | |
| 132 | Candida krusei (Pichia | AIVGASLDLIK | 60S-L24B | A0A099P162 |
| | kudriavzevii) (yeast) | | | |
| 133 | Candida krusei (Pichia | ILDDLVFPTEIVGK | 40S-S7 | A0A099P5W9 |
| | kudriavzevii) (yeast) | | | |
| 134 | Candida krusei (Pichia | FTPGSFTNYITK | 40S-S0 | A0A1Z8JQ17 |
| | kudriavzevii) (yeast) | | | |
| 135 | Candida krusei (Pichia | SAIVQIDATPFK | 40S-S8 | A0A099P980 |
| | kudriavzevii) (yeast) | | | |
| 136 | Candida krusei (Pichia | MIIIAANTPVLR | 60S-L30 | A0A1Z8JHE8 |
| | kudriavzevii) (yeast) | | | |
| 137 | Candida parapsilosis | NSLVHDGLAR | 40S-S12 | G8BBC0 |
| | (yeast) | | | |
| 138 | Candida parapsilosis | AVEVPEQSAYR | 60S-L13 | G8B7X6 |
| | (yeast) | | | |
| 139 | Candida parapsilosis | LLVQQPR | 40S-S27 | G8BKA5 |
| | (yeast) | | | |
| 140 | Candida parapsilosis | IAGVVYHPSNNELVR | 40S-S8 | G8BDI1 |
| | (yeast) | | | |
| 141 | Candida parapsilosis | LISTIDANYLQK | 60S-L8 | G8BAV5 |
| | (yeast) | | | |
| 142 | Candida parapsilosis | ILAESPSPLDLK | 40S-S7 | G8BH01 |
| | (yeast) | | | |
| 143 | Candida parapsilosis | QVVFEIPGETH | 40S-S7 | G8BH01 |
| | (yeast) | | | |
| 144 | Candida parapsilosis | ALAIFVPPPSVVGYR | 40S-S7 | G8BH01 |
| | (yeast) | | | |
| 145 | Candida tropicalis (yeast) | YASSIGR | 60S | C5M6X7 |
| | | | ribosomal | |
| | | | protein | |
| | | | L17-B | |
| 146 | Candida tropicalis (yeast) | LAASGASVVSK | 60S-L24 | C5MI38 |
| 147 | Candida tropicalis (yeast) | NFGIGQSVQPK | 60S | C5M7Z4 |
| | | | ribosomal | |
| | | | protein | |
| | | | L8 | |
| 148 | Candida tropicalis (yeast) | QVVFEIPGENH | 40S-S7 | C5M9K3 |
| 149 | Candida tropicalis (yeast) | SGYTLPANIISNTDVTR | 60S-L24B | C5MC94 |
| 150 | Candida tropicalis (yeast) | ALAVFVPPPSLAAYR | 40S-S7 | C5M9K3 |
| 151 | Capnocytophaga | TAPAAVQLLEAAK | 50S-L11 | A0A2A3N613 |
| | sputigena | | | |
| 152 | Capnocytophaga | NFAEQLVNLTVK | 50S-L7/ | A0A250FJ57 |
| | sputigena | | L12 | |
| 153 | Capnocytophaga | STLGDLEVLQELK | 30S-S1 | A0A2A3N2R7 |
| | sputigena | | | |
| 154 | Capnocytophaga | IMFEVGGVPLDVAK | 50S-L16 | A0A2A3N6A5 |
| | sputigena | | | |
| 155 | Citrobacter freundii and | GNTGENLLGLLEGR | 30S-S4 | A0A1R0FPJ3 |
| | Citrobacter braakii | | | |
| 156 | Citrobacter freundii and | LADVLSAAEAR | 50S-L9 | A0A1R0FR83 |
| | Citrobacter braakii | | | |
| 157 | Citrobacter koseri | LATELALR | 50S-L13 | A0A078LQE7 |
| 158 | Citrobacter koseri | VSVVNNPTGR | 30S-S22 | A0A078LDZ9 |
| 159 | Clostridium perfringens | NALYTPAEALELAVK | 50S-L1 | A0A133N9J9 |
| 160 | Clostridium perfringens | ALLNNMVVGVSQGFSK | 50S-L6 | A0A133N954 |
| 161 | Clostridium perfringens | VLFELSGVDEEK | 50S-L16 | A0A133N961 |
| 162 | Clostridium perfringens | EIETYFGLETLR | 30S-S9 | A0A133N969 |
| 163 | Clostridium perfringens | AIVNILLQEGYLK | 30s-s8 | A0A133N9F3 |
| 164 | Eggerthella lenta | EALVNYALTPFK | 30S-S9 | A0A369N4F7 |
| 165 | Eggerthella lenta | SLLAPLSNK | 50S-L2 | A0A369MGD7 |
| 166 | Eggerthella lenta | LLDAAMGDLR | 50S-L5 | A0A369MT16 |
| 167 | Eggerthella lenta | AADLLVIK | 30S-S4 | A0A369MX18 |
| 168 | Enterobacter asburiae | GISNVSFDR | 50S-L18 | A0A0F1R797 |
| | and Enterobacter | | | |
| | hormachei and | | | |
| | Enterobacter cloacae | | | |
| 169 | Enterobacterales | YLSLLPYTDR | 30S-S18 | A0A37617P4 |
| | common | | | |
| 170 | Enterobacterales | QLGEDPWVAIAK | 30S-S1 | A0A155WS80 |
| | common | | | |
| 171 | Enterobacterales | FTVLISPHVNK | 30S-S10 | A0A5B9AU26 |
| | common | | | |
| 172 | Enterobacterales | VVEPLITLAK | 50S-L17 | A0A2T4HMB2 |
| | common | | | |
| 173 | Enterobacterales | LVADSITSQLER | 30S-S3 | A0A4Q8WTY4 |
| | common | | | |
| 174 | Enterobacterales | FGFTSR | 50S-L15 | A0A078LPA6 |
| | common | | | |
| 175 | Enterobacterales | VANLGSLGDQVNVK | 50S-L9 | A0A376V1H3 |
| | common | | | |
| 176 | Enterobacterales | GGFTVELNGIR | 30S-S1 | A0A2S4QIF8 |
| | common | | | |
| 177 | Enterobacterales | NYITESGK | 30S-S18 | A0A0G3SJ24 |
| | common | | | |
| 178 | Enterobacterales | AVIESENSAER | 30S-S1 | A0A3R7KQS5 |
| | common | | | |
| 179 | Enterobacterales | NMAGSLVR | 50S-L17 | A0A133LFI8 |
| | common | | | |
| 180 | Enterobacterales | FVNILMVDGK | 30S-S7 | A0A078LHA4 |
| | common | | | |
| 181 | Enterococcus common | EGVVLAAFPK | 50S-L24 | A0A1B4XKV5 |
| 182 | Enterococcus common | TQTVLVFAK | 50S-L1 | A0A2S7S2B2 |
| 183 | Enterococcus common | LVDAAYDYMK | 30S-S2 | A0A1B4XQD8 |
| 184 | Enterococcus common | NVELGEYEVGK | 50S-L3 | A0A1B4XKR3 |
| 185 | Enterococcus common | SLGSNTPINVVR | 30S-S5 | A0A1B4XKW0 |
| 186 | Enterococcus common | FLGGIADMPR | 30S-S2 | A0A1V2U816 |
| 187 | Enterococcus common | SVADAISILK | 50S-L22 | A0A133N7C4 |
| 188 | Enterococcus faecalis | EEDESIVVESALQK | 50S-L17 | A0A1B4XKX1 |
| 189 | Enterococcus faecalis | QVLANLSIDTK | 50S-L4 | A0A1B4XKU7 |
| 190 | Enterococcus faecalis | VVVLPAGVEIK | 50S-L6 | A0A4U4BXV7 |
| 191 | Enterococcus faecalis | VSSVEQITALAK | 50S-L10 | A0A1B4XR41 |
| 192 | Enterococcus faecalis | LADAAVSTIEIER | 30S-S3 | A0A1B4XKR8 |
| 193 | Enterococcus faecalis | EVINQPFGVTETK | 30S-S9 | A0A4U3L7L1 |
| 194 | Enterococcus faecalis | FEDGTEVTPVVLK | 50S-L15 | A0A4U3MQ94 |
| 195 | Enterococcus faecium | VYPVAEAVALAK | 50S-L1 | A0A2S7S2B2 |
| 196 | Enterococcus faecium | VSSLEEITALAK | 50S-L10 | A0A3N3L9L2 |
| 197 | Enterococcus faecium | VTIQNLEVVR | 50S-L3 | A0A132P5N2 |
| 198 | Enterococcus faecium | NVQPVLEVK | 30S-S7 | A0A2A7SUB5 |
| 199 | Enterococcus faecium | EENEDIVIESALQK | 50S-L17 | A0A2G0EBS6 |
| 200 | Escherichia coli | YTAAITGAEGK | 30S-S6 | A0A376ZL25 |
| 201 | Escherichia coli | YTQLIER | 30S-S15 | A0A2K3TX98 |
| 202 | Fusobacterium | LVNDELDK | 50S-L2 | A0A4Q2L2G5 |
| | necrophorum and | | | |
| | Fusobacterium | | | |
| | nucleatum | | | |
| 203 | Fusobacterium | SEWAVEGK | 30S-S3 | A0A4Q2L157 |
| | necrophorum and | | | |
| | Fusobacterium | | | |
| | nucleatum | | | |
| 204 | Fusobacterium | AGMYYVNSR | 30S-S2 | A0A2N6THS5 |
| | necrophorum and | | | |
| | Fusobacterium | | | |
| | nucleatum | | | |
| 205 | Fusobacterium | EMTSEDLVVK | 50S-L29 | A0A4Q2KY41 |
| | necrophorum and | | | |
| | Fusobacterium | | | |
| | nucleatum | | | |
| 206 | Fusobacterium | DYNLYLSAR | 50S-L4 | A0A4Q2KY35 |
| | necrophorum and | | | |
| | Fusobacterium | | | |
| | nucleatum | | | |
| 207 | Fusobacterium | NAFAFLR | 50S-L17 | A0A133P930 |
| | necrophorum and | | | |
| | Fusobacterium | | | |
| | nucleatum | | | |
| 208 | Fusobacterium | FQLSLGQLTNTAK | 50S-L29 | A0A4Q2KY41 |
| | necrophorum and | | | |
| | Fusobacterium | | | |
| | nucleatum | | | |
| 209 | Granulicatella adiacens | ELTNDELDR | 30S-S13 | C8NH00 |
| 210 | Granulicatella adiacens | NVAVTTTFGPGVK | 50S-L1 | C8NHK4 |
| 211 | Granulicatella adiacens | AVVELAGISDVTSK | 30S-S5 | C8NH07 |
| 212 | Granulicatella adiacens | IGNKPVVIPAGVTVDLK | 50S-L6 | C8NH09 |
| 213 | Granulicatella adiacens | AYPVQEAIALAK | 50S-L1 | C8NHK4 |
| 214 | Granulicatella adiacens | EAGLEGMDDVFK | 50S-L10 | C8NHK3 |
| 215 | Granulicatella adiacens | AATILYNAFDIVK | 30S-S7 | A0A420YPY2 |
| 216 | Granulicatella adiacens | ELELIGVGYR | 50S-L6 | A0A420YUP9 |
| 217 | Granulicatella adiacens | VAIANILK | 30S-S8 | C8NH10 |
| 218 | Haemophilus influenzae | VNHWVAQGASLSDR | 30S-S16 | A0A0D0IKF5 |
| 219 | Haemophilus influenzae | DVAEAVTAAGVK | 50S-L9 | A0A0K9LCT6 |
| 220 | Haemophilus influenzae | SAAEAAFVEMQK | 30S-S20 | A0A3E1R4M8 |
| 221 | Haemophilus influenzae | ENLQALLAALNK | 50S-L1 | A0A2R3FVP7 |
| 222 | Haemophilus influenzae | AYEINEAIAVLK | 50S-L1 | A0A2R3GAS0 |
| 223 | Hafnia alvei | VSQALETLAYTNK | 50S-L22 | A0A0K0HMQ1 |
| 224 | Hafnia alvei | GIETVLAEIR | 50S-L28 | A0A0K0HUF1 |
| 225 | Hafnia alvei | DVTGIDPVSLIAFDK | 50S-L4 | A0A0K0HNJ7 |
| 226 | Hafnia alvei | SVEELNTELLSLLR | 50S-L29 | A0A0K0HLV5 |
| 227 | Hafnia alvei | ATIDGLASMK | 30S-S5 | A0A0K0HNG2 |
| 228 | Hafnia alvei | MFTIEATAR | 50S-L25 | A0A0M2N8U2 |
| 229 | Hafnia alvei | IGVPFVSGGK | 50S-L21 | A0A0K0HR96 |
| 230 | Klebsiella aerogenes | AQIVSEFGR | 30S-S15 | A0A094X3B8 |
| 231 | Klebsiella aerogenes | DIADAVSAAGVAVAK | 50S-L9 | A0A094WYW2 |
| 232 | Klebsiella oxytoca | AYEDAETVVGIINGK | 30S-S1 | A0A0G3SAC3 |
| 233 | Klebsiella oxytoca | LSDLAHVEGDVIDLNTLK | 50S-L15 | A0A181WY16 |
| 234 | Klebsiella oxytoca | VGFFNPIASATEEGTR | 30S-S16 | A0A0G3S3W6 |
| 235 | Klebsiella pneumoniae | LSDLAHVEGDVVDLNALK | 50S-L15 | A0A0V9RMF1 |
| 236 | Lactobacillus casei | VANVDMNR | 50S-L20 | A0A510WG4 |
| 237 | Lactobacillus casei | AAAPVAAGAAAGGDAAATK | 50S-L7/ | A0A5R8LHU0 |
| | | | L12 | |
| 238 | Lactobacillus casei | VPAGVTVTK | 50S-L6 | A0A0H0YRV1 |
| 239 | Lactobacillus casei | TLLVSQSHTAGR | 50S-L2 | A0A0H0YRU3 |
| 240 | Lactobacillus casei | HESLVVPASK | 30S-S8 | J7M404 |
| 241 | Lactobacillus casei | AYVNEGPTLK | 50S-L22 | J7MDP4 |
| 242 | Lactobacillus casei | DASILELNDLVK | 50S-L7/ | A0A510WWL3 |
| | | | L12 | |
| 243 | Lactobacillus casei | LSSVVASILR | 50S-L13 | A0A510WKM4 |
| 244 | Lactobacillus casei | LNEEDILNWLQK | 30S-S16 | A0A0B8TJY0 |
| 245 | Morganella morganii | NIHNAVEVK | 50S-L6 | A0A0D8LD26 |
| 246 | Morganella morganii | HTGYVGGIK | 50S-L13 | A0A0A5SEE0 |
| 247 | Morganella morganii | VSEGQIVR | 50S-L21 | A0A0A2RI57 |
| 248 | Morganella morganii | ANLAAQIK | 30S-S20 | A0A0A2R704 |
| 249 | Morganella morganii | VGTVTPNVAEAVNNAK | 50S-L1 | A0A2X1V4V4 |
| 250 | Morganella morganii | AANVIGPQIEYAK | 50S-L15 | A0A0A5SG52 |
| 251 | Morganella morganii | SLENYFGR | 30S-S9 | A0A0A5SDF9 |
| 252 | Morganella morganii | VFLSGELTR | 50S-L15 | A0A0A5SG52 |
| 253 | Morganella morganii | VEGVNTLLVK | 50S-L23 | A0A0A2R0G5 |
| 254 | Morganella morganii | LYLGAVATSVR | 30S-S2 | A0A433ZRX6 |
| 255 | Morganella morganii | IGFFNPIAAGK | 30S-S16 | A0A433ZWE8 |
| 256 | Morganella morganii | TVPMFNDALAELNK | 30S-S2 | A0A433ZRX6 |
| 257 | Morganella morganii | ALVNAMVVGVTEGFSK | 50S-L6 | A0A0D8LD26 |
| 258 | Morganella morganii | LYDINEAVALLK | 50S-L1 | A0A1M7NIE9 |
| 259 | Morganella morganii | GNTGENLLSLLEGR | 30S-S4 | A0A0A2R0C2 |
| 260 | Morganella morganii | DVTAIDPVSLIAFGK | 50S-L4 | A0A0A2RFD7 |
| 261 | Morganella morganii | ALLSAFNFPFR | 50S-L5 | A0A433ZXX7 |
| 262 | Morganella morganii | GTVVAIDK | 30S-S1 | A0A0A5SJM7 |
| 263 | Morganella morganii | GATVLPNGTGR | 50S-L1 | A0A2X1V4V4 |
| 264 | Morganella morganii | AGNALPMR | 50S-L2 | A0A2T4HMC2 |
| 265 | Neisseria meningitidis | SGSNVEAAAIVGK | 50S-L18 | A0A0E1IDN6 |
| 266 | Neisseria meningitidis | MFLNTIQPAVGATHAGR | 50S-L15 | A0A1B1X0F4 |
| 267 | Neisseria meningitidis | GLIEFALTEEK | 50S-L3 | A0A0Y5NCX1 |
| 268 | Neisseria meningitidis | DLQVLMGVPVHVNIEEIR | 30S-S3 | X5END1 |
| 269 | Neisseria meningitidis | AGMATILSQLTR | 50S-L4 | A0A378WCJ9 |
| 270 | Neisseria meningitidis | SWVVSELVEK | 30S-S17 | A0A0E1IAC4 |
| 271 | Ochrobactrum anthropi | TAFIALIR | 50S-L2 | A0A011T1K8 |
| 272 | Ochrobactrum anthropi | VLDVLQSEGYIR | 30S-S8 | A0A011VAK8 |
| 273 | Ochrobactrum anthropi | FITIALPR | 50S-L5 | A0A011UHY9 |
| 274 | Pantoea agglomerans | APVVVPAGVEVK | 50S-L6 | A0A1T4SAR2 |
| 275 | Pantoea agglomerans | EGSYVTLR | 50S-L2 | A0A379AA07 |
| 276 | Pantoea agglomerans | GLPTPVVITVYSDR | 50S-L11 | A0A1T4SCA6 |
| 277 | Pantoea agglomerans | QPLELLDLVGK | 30S-S9 | A0A2A7V6D3 |
| 278 | Parvimonas micra | ILDALTIDAFSTK | 50S-L4 | A0A0B4S0G8 |
| 279 | Parvimonas micra | ITGDALVMMLETR | 30S-S4 | A0A3B7DNI5 |
| 280 | Parvimonas micra | VLFEMSGVPVDVAR | 50S-L16 | A0A3B7DDP4 |
| 281 | Parvimonas micra | ALLELLGMPFK | 50S-L5 | A0A3B7DGA2 |
| 282 | Proteus mirabilis | QYEINEAVALLK | 50S-L1 | A0A1Z1SRZ3 |
| 283 | Proteus mirabilis | EAFQLAAAK | 50S-L16 | A0A2X2EDT7 |
| 284 | Proteus mirabilis | VDFNEAQLK | 50S-L1 | A0A1Z1SRZ3 |
| 285 | Proteus mirabilis | IVAEFGR | 30S-S15 | A0A5F0SX42 |
| 286 | Proteus mirabilis | ATMAGLGLR | 50S-L30 | A0A1Z1T0D6 |
| 287 | Proteus vulgaris | LATLPTYDEAIAR | 50S-L10 | A0A2J9L748- |
| | | | | 1 |
| 288 | Proteus vulgaris | QYEITEAVALLK | 50S-L1 | A0A379F9S2 |
| 289 | Proteus vulgaris | VDFNETQLK | 50S-L1 | A0A379F9S2 |
| 290 | Pseudomonas aeruginosa | AALSAVVADAR | 50S-L10 | A0A072ZCB6 |
| 291 | Pseudomonas aeruginosa | IIQQIEAEQMNK | 50S-L19 | A0A5K1SQJ0 |
| 292 | Pseudomonas aeruginosa | LPAGVEIK | 50S-L6 | A0A2V3F3S9 |
| 293 | Pseudomonas aeruginosa | VHPSVEVIQDSGELR | 50S-L6 | A0A2V3F3S9 |
| 294 | Pseudomonas aeruginosa | YTALIGR | 30S-S15 | A0A071L3R7 |
| 295 | Pseudomonas aeruginosa | STPAAVLLK | 50S-L11 | A0A1C7BKP7 |
| 296 | Pseudomonas aeruginosa | VEGDVVSLQTLK | 50S-L15 | A0A5E5R746 |
| 297 | Pseudomonas aeruginosa | IGLPVVEGAK | 50S-L21 | A0A2R3IZT6 |
| 298 | Pseudomonas aeruginosa | VTVQSLEIVR | 50S-L3 | A0A072ZBZ2 |
| 299 | Pseudomonas aeruginosa | LVVGGVNLIK | 50S-L24 | A0A1C7B7C3 |
| 300 | Pseudomonas aeruginosa | AGDVVAVR | 30S-S4 | A0A072ZDF7 |
| 301 | Pseudomonas | TVLNQQAGK | 50S-L29 | A0A178LBZ8 |
| | oryzihabitans and | | | |
| | Pseudomonas putida | | | |
| 302 | Pseudomonas | STGEVHLR | 50S-L32 | A0A160GMP7 |
| | oryzihabitans and | | | |
| | Pseudomonas putida | | | |
| 303 | Pseudomonas | SVLEAENSAER | 30S-S1 | A0A2Z5AD07 |
| | oryzihabitans and | | | |
| | Pseudomonas putida | | | |
| 304 | Pseudomonas | ALDAIAPLVEVK | 30S-S7 | A0A178LAS2 |
| | oryzihabitans and | | | |
| | Pseudomonas putida | | | |
| 305 | Pseudomonas | FGFVSLK | 50S-L15 | A0A2Z5ADV2 |
| | oryzihabitans and | | | |
| | Pseudomonas putida | | | |
| 306 | Raoultella | ALLNSVVIGVTEGFTK | 50S-L6 | A0A4R2XU01 |
| | ornithinolytica | | | |
| 307 | Raoultella | SATGLGLK | 50S-L7/ | A0A038CMN2 |
| | ornithinolytica | | L12 | |
| 308 | Rothia dentocariosa | QAQEVIAEQATR | 30S-S2 | A0A2A8D5U0 |
| 309 | Rothia dentocariosa | AHETLAATGVNPDTR | 30S-S13 | A0A2A8D8A9 |
| 310 | Rothia dentocariosa | HIMVDGVR | 30S-S4 | A0A269YJQ1 |
| 311 | Rothia dentocariosa | IVYGALEGVEK | 30S-S7 | A0A2A8D8D8 |
| 312 | Rothia dentocariosa | AEAAEEPMAAWER | 30S-S2 | A0A2A8D5U0 |
| 313 | Rothia dentocariosa | LIDVVDPTPK | 30S-S10 | A0A2A8D817 |
| 314 | Rothia dentocariosa | IIDIDMDR | 30S-S1 | A0A269YL77 |
| 315 | Rothia dentocariosa | VLNLNESVMR | 30S-S6 | A0A269YH40 |
| 316 | Rothia dentocariosa | ELSLVEVSEFVK | 50S- | A0A211VKP6 |
| | | | L7/L12 | |
| 317 | Rothia dentocariosa | TADPVTVGMVNTVAHLVK | 50S-L30 | A0A2A8D8M0 |
| 318 | Rothia dentocariosa | LSIEELIAAFK | 50S-L7/ | A0A211VKP6 |
| | | | L12 | |
| 319 | Rothia dentocariosa | GSVILPEAITPK | 30S-S16 | A0A3S5C0X3 |
| 320 | Salmonella enterica | DIADAVTAAGVDVAK | 50S-L9 | A0A100PT80 |
| 321 | Salmonella enterica | VAVFTQGPNAEAAK | 50S-L1 | A0A447PRH1 |
| 322 | Serratia marcescens | SLDTDGYR | 50S-L3 | A0A1C3HK02 |
| 323 | Serratia marcescens | TIHDAVEVK | 50S-L6 | A0A080UYA0 |
| 324 | Serratia marcescens | INELGSVTIASK | 50S-L9 | A0A086FJA0 |
| 325 | Serratia marcescens | QEANALTFAPR | 50S-L6 | A0A080UYA0 |
| 326 | Serratia marcescens | DVAGIDPVSLIAFDK | 50S-L4 | A0A379YCZ8 |
| 327 | Serratia marcescens | DGSYVTLR | 50S-L2 | A0A0M5M078 |
| 328 | Staphylococcus aureus | SGVMEGNVITAEEVK | 50S-L10 | A0A0H2HJN6 |
| | and Staphylococcus | | | |
| | argenteus | | | |
| 329 | Staphylococcus aureus | IIEQIGTYNPTSANAPEIK | 30S-S16 | A0A380C4X4 |
| | and Staphylococcus | | | |
| | argenteus | | | |
| 330 | Staphylococcus aureus | SQSVLVFAK | 50S-L1 | A0A2X2KBV1 |
| | and Staphylococcus | | | |
| | argenteus | | | |
| 331 | Staphylococcus aureus | VLELVGVGYR | 50S-L6 | A0A380ENQ7 |
| | and Staphylococcus | | | |
| | argenteus | | | |
| 332 | Staphylococcus aureus | MAVEEIFNVK | 50S-L23 | A0A380DT12 |
| | and Staphylococcus | | | |
| | argenteus | | | |
| 333 | Staphylococcus aureus | NYAVEATPGNLK | 50S-L9 | A0A5C8X5P0 |
| | and Staphylococcus | | | |
| | argenteus | | | |
| 334 | Staphylococcus aureus | AGIDINR | 50S-L20 | A0A077VMP6 |
| | and Staphylococcus | | | |
| | argenteus | | | |
| 335 | Staphylococcus aureus | VVVEGVNIMK | 50S-L24 | A0A380CL60 |
| | and Staphylococcus | | | |
| | argenteus | | | |
| 336 | Staphylococcus aureus | ILEEANVSADTR | 30S-S13 | W8U8U6 |
| | and Staphylococcus | | | |
| | argenteus | | | |
| 337 | Staphylococcus aureus | VDEALALK | 30S-S16 | A0A380C4X4 |
| | and Staphylococcus | | | |
| | argenteus | | | |
| 338 | Staphylococcus coagulase | AVLELAGITDILSK | 30S-S5 | A0A2T4LTV0 |
| | negative | | | |
| 339 | Staphylococcus coagulase | VIFLDTTTDFK | 50S-L31 | A0A2T7BSB7 |
| | negative | | type B | |
| 340 | Staphylococcus coagulase | MFAIIETGGK | 50S-L21 | A0A2T4LQZ0 |
| | negative | | | |
| 341 | Staphylococcus coagulase | IDEELALK | 30S-S16 | A0A0U1EFK2 |
| | negative | | | |
| 342 | Staphylococcus coagulase | SLLQPLPK | 50S-L2 | A0A2N6QET1 |
| | negative | | | |
| 343 | Staphylococcus | VIFLDTTTNYK | 50S-L31 | A0A2K4DRZ8 |
| | pettenkorferi | | type B | |
| 344 | Staphylococcus | ALINNMIQGVK | 50S-L6 | A0A4Y9KNP3 |
| | saprophyticus | | | |
| 345 | Staphylococcus | ILYSAFDLVK | 30S-S7 | A0A4Y9KN52 |
| | saprophyticus | | | |
| 346 | Stenotrophomonas | AYAFEDAINILK | 50S-L1 | A0A2J0URS2 |
| | maltophilia | | | |
| 347 | Stenotrophomonas | DFSEDLVHQVVVAYR | 50S-L4 | A0A4S2CXM0 |
| | maltophilia | | | |
| 348 | Stenotrophomonas | DTAEVLLYALDK | 50S-L15 | A0A2W5HNQ9 |
| | maltophilia | | | |
| 349 | Streptococcus agalactiae | TQLESETTR | 50S-L9 | A0A380IJI9 |
| 350 | Streptococcus agalactiae | GQVPGVTK | 30S | R4Z8C7 |
| | | | ribosomal | |
| | | | protein | |
| | | | S14 type | |
| | | | Z | |
| 351 | Streptococcus agalactiae | EGASEAEANEIK | 50S-L7/ | A0A0H1HYJ3 |
| | | | L12 | |
| 352 | Streptococcus agalactiae | SMVALEAGK | 50S-L23 | A0A5N0LG39 |
| 353 | Streptococcus agalactiae | GLTVEQDTNLR | 50S-L10 | A0A076YXU6 |
| 354 | Streptococcus agalactiae | FTSVEEINALAK | 50S-L10 | A0A076YXU6 |
| 355 | Streptococcus agalactiae | LEAAGASVTLK | 50S-L7/ | A0A0H1HYJ3 |
| | | | L12 | |
| 356 | Streptococcus agalactiae | DVLSAGQEVTVK | 30S-S1 | A0A3P1APL0 |
| 357 | Streptococcus agalactiae | AIVDNAPSVIK | 50S-L7/ | A0A0H1HYJ3 |
| | | | L12 | |
| 358 | Streptococcus agalactiae | GTHIYPGANVGR | 50S-L27 | X5K262 |
| 359 | Streptococcus agalactiae | SDIPEFR | 50S-L19 | R4Z9H3 |
| 360 | Streptococcus bovis and | FDETTGDYSR | 50S-L32 | A0A0W7V274 |
| | related | | | |
| 361 | Streptococcus bovis and | AEDVAALR | 30S-S5 | A0A060RIT2 |
| | related | | | |
| 362 | Streptococcus bovis and | TAEFANVLSALNVDSK | 50S-L4 | A0A380KMA3 |
| | related | | | |
| 363 | Streptococcus bovis and | GTAASIVYDAFEQIK | 30S-S7 | A0A135YPT7 |
| | related | | | |
| 364 | Streptococcus bovis and | LTAPSVK | 50S-L32 | A0A0U3E3C5 |
| | related | | | |
| 365 | Streptococcus bovis and | TVAALGLGK | 50S-L30 | A0A211YE03 |
| | related | | | |
| 366 | Streptococcus bovis and | FIQTELADASVSR | 30S-S3 | A0A368UCF0 |
| | related | | | |
| 367 | Streptococcus bovis and | EVVPAENR | 30S-S1 | A0A3E2SH26 |
| | related | | | |
| 368 | Streptococcus bovis and | NEIASENFDEATEK | 50S-L17 | A0A0U3EVP9 |
| | related | | | |
| 369 | Streptococcus bovis and | IAGVDIPNEK | 30S-S13 | A0A380K260 |
| | related | | | |
| 370 | Streptococccus common | DFHGVPTK | 50S-L5 | A0A0C1K3D8 |
| 371 | Streptococccus common | NGIHVIDLQQTVK | 30S-S2 | A0A0E1EJH5 |
| 372 | Streptococcus common | VLVFAR | 50S-L1 | A0A098ZG22 |
| 373 | Streptococcus common | SLGSNTPINIVR | 30S-S5 | A0A564S853 |
| 374 | Streptococcus common | MIEGTAR | 50S-L11 | A0A1L7MTN8 |
| 375 | Streptococcus common | INVADSR | 30S-S16 | A0A0F5MHU4 |
| 376 | Streptococcus common | AIITLTADSK | 50S-L23 | A0A5N0LG39 |
| 377 | Streptococcus common | LGLATTR | 30S-S4 | A0A4V6L8K5 |
| 378 | Streptococcus | VEAGQVISVR | 30S-S4 | A0A2X2YUF3 |
| | dysgalactiae | | | |
| 379 | Streptococcus | VINDFAK | 50S-L10 | A0A2X2UQ03 |
| | dysgalactiae | | | |
| 380 | Streptococcus | VVVEGVGMIK | 50S-L24 | A0A2Z6G1V6 |
| | dysgalactiae | | | |
| 381 | Streptococcus | VLEWLAK | 30S-S16 | A0A2X2WJV0 |
| | dysgalactiae | | | |
| 382 | Streptococcus other | AADGQTVTGGSILYR | 50S-L27 | A0A380JJ90 |
| | streptococcus | | | |
| 383 | Streptococcus other | QAVEAAFEGVK | 50S-L23 | A0A081QM54 |
| | streptococcus | | | |
| 384 | Streptococcus other | IVSGPEADIK | 50S-L2 | A0A0F2CL38 |
| | streptococcus | | | |
| 385 | Streptococcus other | FVAVDSLSFTAPK | 50S-L4 | A0A0F2CNQ7 |
| | streptococcus | | | |
| 386 | Streptococcus other | IQIFEGVVIAR | 50S-L19 | A0A1L7MUI0 |
| | streptococcus | | | |
| 387 | Streptococcus | AAGDYEGLSK | 30S-S14 | J1P244 |
| | pneumoniae | | | |
| 388 | Streptococcus | FVGQEFDTK | 30S-S1 | A0A4M3JJF9 |
| | pneumoniae | | | |
| 389 | Streptococcus pyogenes | SAEAAIIAK | 50S-L15 | A0A4U7HLR0 |
| 390 | Streptococcus pyogenes | VDPGQVISVR | 30S-S4 | A0A4Q1PT68 |
| 391 | Streptococcus pyogenes | VINDFTK | 50S-L10 | A0A4V6EC94 |
| 392 | Streptococcus pyogenes | VIVEGVGMIK | 50S-L24 | A0A4V6ELU1 |
| 393 | Citrobacter amalonaticus | AADMTGADIEAMTR | 50S-L11 | A0A2S4RQD3 |
| | and Citrobacter farmeri | | | |
| | and Citrobacter sedlakii | | | |
| | and Citrobacter koseri | | | |
| 394 | Citrobacter amalonaticus | LADVLAAANAR | 50S-L9 | A0A381GFD3 |
| | and Citrobacter farmeri | | | |
| | and Citrobacter sedlakii | | | |
| | and Citrobacter koseri | | | |
| 395 | Citrobacter amalonaticus | INALETVTITSK | 50S-L9 | A0A381GFD3 |
| | and Citrobacter farmeri | | | |
| 396 | Bacteroides fragilis and | MEVVNALGR | 30S-S9 | A0A0K6BYT6 |
| | Bacteroides | | | |
| | thetaiotamicron and | | | |
| | Bacteroides vulgatus | | | |
| 397 | Bacteroides fragilis and | YLTPPSVDVK | 30S-S18 | A0A0P0F6X2 |
| | Bacteroides | | | |
| | thetaiotamicron and | | | |
| | Bacteroides vulgatus | | | |
| 398 | Citrobacter braakii and | YTAAITGAEGTIHR | 30S-S6 | A0A1R0FR01 |
| | Citrobacter freundii and | | | |
| | Citrobacter youngae and | | | |
| | Citrobacter werkmanii | | | |
| | and Citrobacter | | | |
| | portucalensis and | | | |
| | Citrobacter cronae | | | |
| 399 | Citrobacter braakii and | TLNDAVAVNHADNALTFGPR | 50S-L6 | A0A1R0FPQ2 |
| | Citrobacter freundii and | | | |
| | Citrobacter youngae and | | | |
| | Citrobacter werkmanii | | | |
| | and Citrobacter | | | |
| | portucalensis and | | | |
| | Citrobacter cronae | | | |
| 400 | Citrobacter braakii and | AGDQVQSGVDAAIK | 50S-L2 | A0A1R0FPH1 |
| | Citrobacter cronae | | | |
| 401 | Klebsiella pneumoniae | YTGAITAAAGTIHR | 30S-S6 | A0A377UTB6 |
| 402 | Proteus mirabilis | AAFAALVEK | 50S-L20 | B4ETK9 |
| 403 | Proteus vulgaris and | SIVVAIDR | 30S-S17 | A0A0J1CB87 |
| | Proteus columbae and | | | |
| | Proteus penneri and | | | |
| | Proteus terrae | | | |
| 404 | Proteus vulgaris and | INALGSVTISSK | 50S-L9 | A0A617CX33 |
| | Proteus columbae and | | | |
| | Proteus penneri and | | | |
| | Proteus terrae | | | |
| 405 | Providencia rettgeri and | AANVVGIQIEYAK | 50S-L15 | A0A1B8SN57 |
| | Providencia stuartii | | | |
| 406 | Providencia rettgeri and | AGDQIQSGVDSAIK | 50S-L2 | A0A2A5PZW6 |
| | Providencia stuartii | | | |
| 407 | Providencia rettgeri and | DMVESAPATIK | 50S-L7/ | A0A2A5Q0U7 |
| | Providencia stuartii | | L12 | |
| 408 | Providencia rettgeri and | AAAFEGELIQAK | 50S-L10 | A0A1J0E2F3 |
| | Providencia stuartii | | | |
| 409 | Providencia rettgeri and | LSDFAAVEGDVIDLNALK | 50S-L15 | A0A1B8SN57 |
| | Providencia stuartii | | | |
| 410 | Staphylococcus capitis | SLELVGVGYR | 50S-L6 | A0A4U9T7X3 |
| 411 | Staphylococcus capitis | YNSEVTENLVK | 50S-L5 | A0A0U1E9T8 |
| | and Staphylococcus | | | |
| | caprae | | | |
| 412 | Staphylococcus capitis | TGVMEGSVISAEEVK | 50S-L10 | A0A0S4MFX3 |
| | and Staphylococcus | | | |
| | caprae and | | | |
| | Staphylococcus | | | |
| | haemolyticus | | | |
| 413 | Staphylococcus capitis | SGAEVSGPIPLPTEK | 30S-S10 | A0A2K0A6A6 |
| | and Staphylococcus | | | |
| | haemolyticus | | | |
| 414 | Staphylococcus caprae | ELVDNAPK | 50S-L7/ | A0A657ZQ88 |
| | and Staphylococcus | | L12 | |
| | haemolyticus and | | | |
| | Staphylococcus hominis | | | |
| 415 | Staphylococcus | APGSVGMASDASK | 50S-L3 | A0A0N1MT05 |
| | epidermidis | | | |
| 416 | Staphylococcus | HIGSPNEVLEPGQQVNVK | 30S-S1 | A0A0N0LVM7 |
| | epidermidis | | | |
| 417 | Staphylococcus | ILGIDEDNER | 30S-S1 | A0A7I0BF73 |
| | epidermidis | | | |
| 418 | Staphylococcus hominis | YYSVEEAIK | 50S-L1 | A0A4Q9WUN2 |
| 419 | Staphylococcus hominis | ILFEIAGVSEDVAR | 50S-L16 | A0A1L8Y808 |
| 420 | Staphylococcus | ILGVDEDNER | 30S-S1 | A0A4Q9WAN7 |
| | lugdunensis | | | |
| 421 | Staphylococcus | VPAVVYGYSTK | 50S-L25 | A0A133PZL6 |
| | lugdunensis | | | |
| 422 | Proteus vulgaris and | LAETLAAAEAR | 50S-L9 | A0A1Z1SQ72 |
| | Proteus columbae and | | | |
| | Proteus penneri and | | | |
| | Proteus terrae and | | | |
| | Proteus mirabilis | | | |
| 423 | Proteus vulgaris and | LYLTAAATAVR | 30S-S2 | A0A617D3V2 |
| | Proteus columbae and | | | |
| | Proteus penneri and | | | |
| | Proteus terrae and | | | |
| | Proteus mirabilis | | | |

The method of the invention allows in particular the identification of the microorganisms belonging to the following groups:
Enterobacterales
Acinetobacter
Enterococcus
Candida
Staphylococcus, and
Streptococcus.

The group of Enterobacterales called Enterobacterales_common includes 32 species: Citrobacter freundii, Citrobacter braakii, Citrobacter koseri, Citrobacter youngae, Citrobacter werkmanii, Citrobacter portucalensis, Citrobacter cronae, Citrobacter amalonaticus, Citrobacter farmeri, Citrobacter sedlakii, Citrobacter koseri, Enterobacter asburiae, Enterobacter cloacae, Enterobacter hormachei, Escherichia Coli, Hafnia alvei, Klebsiella aerogenes, Klebsiella oxytoca, Klebsiella pneumoniae, Morganella_morganii, Pantoea agglomerans, Proteus mirabilis, Proteus vulgaris, Proteus columbae, Proteus penneri, Proteus terrae, Raoultella ornithinolytica, Salmonella enterica, Serratia marcescens, Providencia rettgeri and Providencia stuartii.

Peptides for this Enterobacterales_common group are common to all species in the group.

The Acinetobacter group called Acinetobacter_common includes 4 Acinetobacter: Acinetobacter baumannii, Acinetobacter lwoffii, Acinetobacter ursingii and Acinetobacter pittii.

Peptides for this Acinetobacter_common group are common to all species in the group.

The Enterococcus group called Enterococcus_common includes 2 Enterococcus: Enterococcus faecium and Enterococcus faecalis.

Peptides for this Enterococcus_common group are common to all species in the group.

The group of Candida called Candida_common includes 7 Candida: Candida albicans, Candida auris, Candida glabrata, Candida kefyr, Candida krusei, Candida tropicalis and Candida parapsilosis.

Peptides for this Candida_common group are common to all species in the group.

The Staphylococcus_coagulase_negative group comprises 10 Coagulase Negative Staphylococcus: Staphylococcus capitis, Staphylococcus caprae, Stapylococcus cohnii, Staphylococcus epidermidis, Stahylococcus haemolyticus, Staphylococcus hominis, Staphylococcus lugdunensis, Staphylococcus pettenkorferi, Staphylococcus saprophyticus, Staphylococcus warneri.

Peptides for this Staphylococcus_coagulase_negative group are common to all species in the group.

The Streptococcus_common group includes 17 Streptococcus: Streptococcus agalactiae, Streptococcus dysgalactiae, Streptococcus pneunomiae, Streptococcus pyogenes, Streptococcus gallolyticus subsp. Gallolyticus, Streptococcus pasteurianus (Streptococcus gallolyticus spp. Pasteurianus), Streptococcus infantarius spp. Coli (or Streptococcus lutetiensis), Streptococcus infantarius subsp. Infantarius, Streptococcus mitis, Streptococcus oralis, Streptococcus salivarius, Streptococcus anginosus, Streptococcus parasanguinis, Streptococcus constellatus, Streptococcus sanguinis, Streptococcus gordonii, Streptococcus intermedius.

Peptides for this Streptococcus_common group are common to all species in the group.

The Streptococcus_bovis_and_related group includes 4 Streptococcus: Streptococcus gallolyticus subsp. Gallolyticus, Streptococcus pasteurianus (Streptococcus gallolyticus spp. Pasteurianus), Streptococcus infantarius spp. Coli (or Streptococcus lutetiensis), Streptococcus infantarius subsp. Infantarius.

Peptides for this Streptococcus_bovis_and_related group are common to all species in the group.

The Streptococcus_other_streptococcus group includes 9 Streptococcus: Streptococcus mitis, Streptococcus oralis, Streptococcus salivarius, Streptococcus anginosus, Streptococcus parasanguinis, Streptococcus constellatus, Streptococcus sanguinis, Streptococcus gordonii, Streptococcus intermedius.

Peptides for this Streptococcus_other_streptococcus group are common to all species in the group.

### Mass Spectrometer

The present method uses a mass spectrometer coupled to a liquid separation device and to processing means.

In a specific embodiment of the invention, the mass spectrometer is a tandem mass spectrometer.

A tandem mass spectrometer is capable of multiple rounds of mass spectrometry, usually separated by some form of molecule fragmentation. Tandem MS can also be done in a single mass analyzer over time, as in a quadrupole ion trap. There are various methods for fragmenting molecules for tandem MS, including collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD) and surface-induced dissociation (SID).

In another embodiment of the invention, the mass spectrometer uses one of the following technologies: PRM (Parallel Reaction Monitoring), MRM (Multi Reaction Monitoring), DIA (Data Independent Acquisition) or SWATH (Sequential Window Acquisition of all THeoretical fragment ion spectra mass spectrometry).

These techniques are well known by the person skilled in the art.

The present invention also concerns a system for the implementation of the method described above, comprising a mass spectrometer coupled to a liquid separation device, and comprising processing means adapted for implementing steps (e) to (h), in particular adapted:
to receive data concerning a plurality of transitions to be used to monitor the mixture of peptides,
to assign the plurality of transitions into two or more contiguous groups of transitions, into said predefined list of transitions,
to monitor at least one sentinel transition in each group of the two or more contiguous groups,
to start the monitoring of at least one transition in a next contiguous group, when the signal of at least one sentinel transition of a group is detected by the mass spectrometer, and
optionally, to generate a chromatogram or an electropherogram.

In a classical way, the system comprises a data processing module, i.e. a computer such as a processor, a microprocessor, a controller, a microcontroller, an FPGA, etc. This computer is adapted to execute code instructions to implement, if necessary, part of the data processing which is presented above. This computer is adapted to execute code instructions to implement part of the data processing that is presented above.

The system also includes a data storage module (a memory, for example flash) and advantageously a user interface (typically a screen), and biometric acquisition means.

### Sentinel Compounds

The present invention uses the technology described in the patent EP 3 384 517, herein designated as the “Sentinel” acquisition mode, that allows great multiplexing capacity.

This methodology involves detection of “sentinel compounds” with a tandem mass spectrometer, which is more precisely a method for triggering a group of multiple reaction monitoring (MRM) transitions from a series of contiguous groups when at least one sentinel transition of the group is detected as part of a previous group, comprising:
separating one or more compounds from a sample using a separation device;
ionizing the separated one or more compounds received from the separation device using an ion source, producing an ion beam of one or more precursor ions;
receiving the ion beam from the ion source using a tandem mass spectrometer and, for each cycle of a plurality of cycles, executing on the ion beam a series of MRM precursor ion to product ion transitions read from a list using the tandem mass spectrometer, wherein for each transition of the series, the tandem mass spectrometer selects and fragments a precursor ion of the each transition and mass analyzes a small mass-to-charge ratio (m/z) range around the m/z of a product ion of the each transition to determine if the product ion of the each transition is detected;
receiving a plurality of MRM transitions to be used to monitor the sample using a processor;
   characterized in that the method further comprises:
dividing the plurality of MRM transitions into two or more contiguous groups of MRM transitions so that different groups can be monitored separately during the plurality of cycles using the processor;
selecting at least one sentinel transition in each group of the two or more contiguous groups that identifies a next group of the two or more contiguous groups that is to be monitored using the processor;
placing a first group of the two or more contiguous groups on the list of the tandem mass spectrometer using the processor; and
when at least one sentinel transition of the first group is detected by the tandem mass spectrometer, placing a next group of the two or more contiguous groups identified by the sentinel transition on the list using the processor.

Application of this method to the detection of microorganisms is described in the present specification.

The sentinel compound used in the present method may be chosen among the following compounds:
peptides issued from step (a) of cleavage of proteins of the microorganism, also designated as “endogenous peptides”; in this case, the method is hereafter designated as “Sentinel-endogenous” method;
peptides issued from autocleavage (self-digestion) of the trypsin enzyme, or peptides issued from the cleavage of proteins or peptides introduced into the sample, or exogenous introduced peptides, also designated as “exogenous peptides”; in this case, the method is hereafter designated as “Sentinel-exogenous” method;
other compounds.

Each of the implementation of the method is presented in more details in the examples section.

During the process of the invention, in steps (g) and (h), at least one sentinel transition associated with one sentinel compound is monitored, using the processor; and when the signal of at least one sentinel transition of a group is detected with the mass spectrometer, the monitoring of at least one sentinel transition in a next contiguous group starts, while the monitoring of the transitions of the preceding group is stopped, using the processor.

Sentinel compounds are selected as having the latest expected retention time in their group of transitions. In other words, they are adapted to stop the monitoring of transitions associated to a peptides group with earlier retention times than the sentinel, and thus to initiate monitoring of transitions associated to the peptides group with later retention times than the sentinel, using the processor.

### Group of Peptides

In another aspect, the present invention relates to a group of peptides adapted for the implementation of the method as described above, wherein said peptides are issued from ribosomal proteins of microorganisms, comprise between 6 and 20 amino acids, and are decomplexed with a mobile phase comprising less than 40% of acetonitrile, on a reverse phase column, preferentially on an octadecyl reverse phase column.

Furthermore, these peptides are specific of a genus and/or a species of a microorganism, and therefore are useful for its identification.

This group of peptides may comprise at least two, three, four, five, six, seven, eight, nine ten, twenty, thirty, forty, fifty, sixty, seventy, eighty, ninety, hundred, two hundred, three hundred or four hundred distinct peptides.

In a specific embodiment of the invention, the group of peptides comprises at least one peptide presenting a peptide sequence selected from SEQ ID NO. 1 to SEQ ID NO. 423.

Advantageously, this group of peptides comprise at least two, three, four, five, six, seven, eight, nine ten, twenty, thirty, forty, fifty, sixty, seventy, eighty, ninety, hundred, two hundred, three hundred or four hundred distinct peptides chosen among the group of peptides having a peptide sequence selected from SEQ ID NO. 1 to SEQ ID NO. 423.

Particular groups of peptides according to the invention are the following:
peptides specific to Enterobacterales, in particular presenting a sequence chosen among SEQ ID NO. 155 to 158, SEQ ID NO. 168 to 180, SEQ ID NO. 200 to 201, SEQ ID NO. 223 to 229, SEQ ID NO. 245 to 264, SEQ ID NO. 274 to 277, SEQ ID NO. 282 to 289, SEQ ID NO. 306 to 307, SEQ ID NO. 320 to 327, SEQ ID NO. 393 to 395, SEQ ID NO. 398 to 409 and SEQ ID NO. 422 to 423;
peptides specific to Acinetobacter, in particular presenting a sequence chosen among SEQ ID NO. 13 to SEQ ID NO. 42;
peptides specific to Enterococcus, in particular presenting a sequence chosen among SEQ ID NO. 181 to SEQ ID NO. 199;
peptides specific to Candida, in particular presenting a sequence chosen among SEQ ID NO. 81 to SEQ ID NO. 150;
peptides specific to Staphylococcus, in particular presenting a sequence chosen among SEQ ID NO. 328 to SEQ ID NO. 345 and SEQ ID NO. 410 to SEQ ID NO. 421;
peptides specific to Streptococcus, in particular presenting a sequence chosen among SEQ ID NO. 349 to SEQ ID NO. 392;
peptides specific to Pseudomonas aeruginosa, in particular presenting a sequence chosen among SEQ ID NO. 290 to SEQ ID NO. 300; or
peptides specific to other genus/species, in particular presenting a sequence chosen among SEQ ID NO. 1 to SEQ ID NO. 12, SEQ ID NO. 43 to 80, SEQ ID NO. 151 to 154, SEQ ID NO. 159 to 167, SEQ ID NO. 202 to 222, SEQ ID NO. 230 to 244, SEQ ID NO. 265 to 273, SEQ ID NO. 278 to 281, SEQ ID NO. 301 to 305, SEQ ID NO. 308 to 319, SEQ ID NO. 346 to 348 and SEQ ID NO. 396 to 397.

In a specific embodiment, the group of peptides comprises all the peptides listed in table 1.

In another embodiment, the group of peptides consists of the 423 peptides as listed in table 1.

These groups of peptides according to the invention may be used in any method or process for the identification of at least one microorganism in a sample.

The present invention also relates to the use of one of this group of peptides as defined above, for the identification of at least one microorganism in a sample, in particular in a biological sample, more particularly in a human blood sample.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### Example 1: Construction of a Sentinel-MRM Method with Endogenous Peptides as Group Triggers

To identify a microorganism, it is possible, for example, to use endogenous peptides as group triggers, i.e., as sentinel compounds.

For example, it is possible to build a Sentinel-MRM method as described below.

The specific peptides of each micro-organism are divided into the following 8 major groups:
a) An Enterobacterales group to identify 32 Enterobacterales;
b) A Pseudomonas aeruginosa group to identify Pseudomonas aeruginosa;
c) A Staphylococcus aureus-argenteus group to identify Staphylococcus aureus or argenteus;
d) An Acinetobacter group to identify 4 Acinetobacter;
e) An Enterococcus group to identify 2 Enterococcus;
f) A Candida group to identify 7 Candida;
g) A Streptococcus and others group to identify 17 Streptococcus and 4 other species;
h) A “other species” group identifying 34 species including 10 coagulase negative Staphylococcus.

Each group (except group h) comprises peptides specific to each species of the group and peptides common to all species of the group.

For groups a to f, two Sentinel peptides are able to trigger the group. These two Sentinel peptides are strictly specific to the desired genus and common to all the species of the group.

For example, for the group a) Enterobacterales, the Sentinel peptides are two peptides common to all Enterobacterales and present only in Enterobacterales species, meaning they are not found in any of the other groups.

Consider the example of a sample containing the pathogen Acinetobacter baumannii, during the Sentinel-MRM analysis of the sample, group d) and only group d) will be triggered and the transitions of the Acinetobacter peptides will be monitored. As the group also includes peptides specific to Acinetobacter baumannii, the identification will be successful.

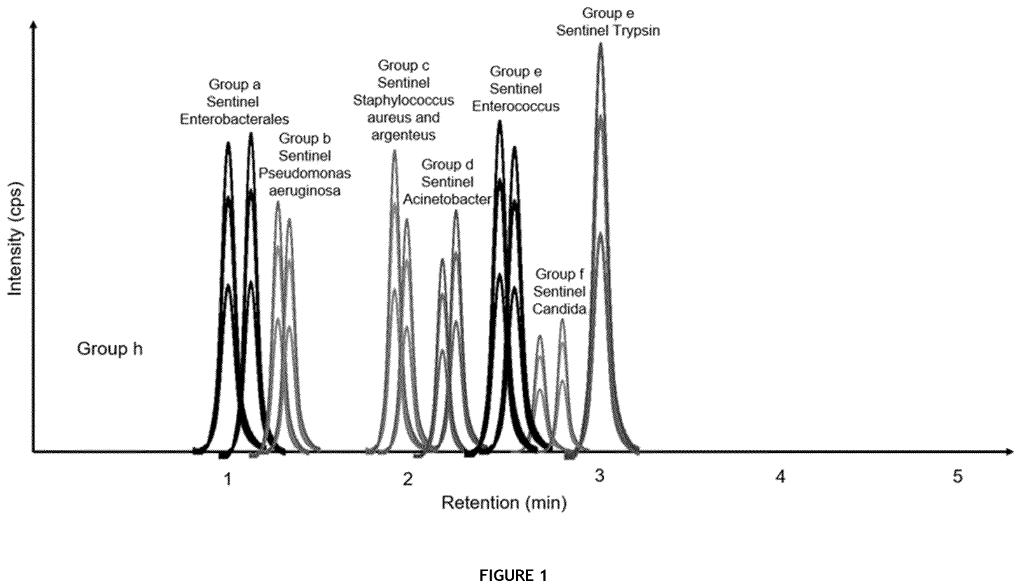

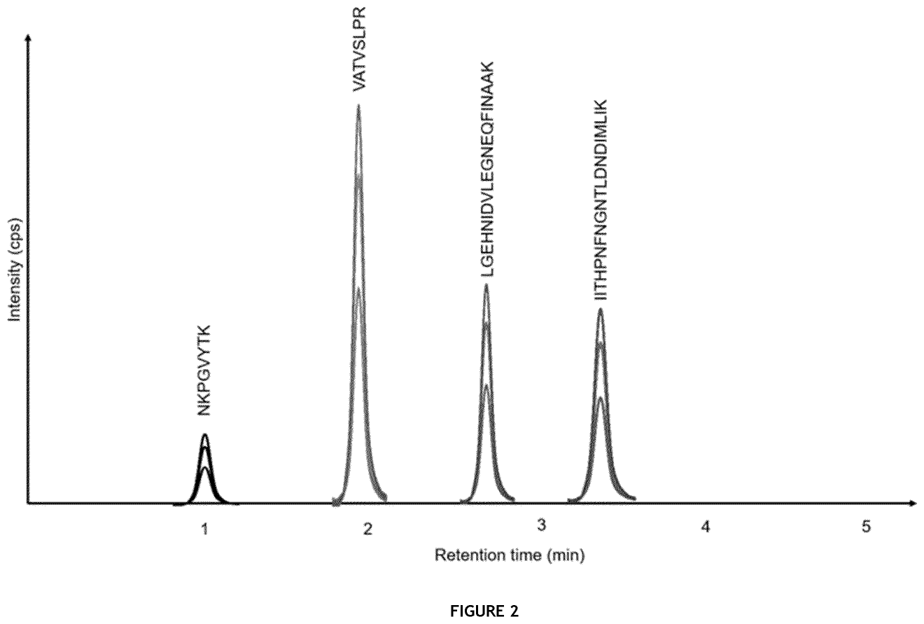

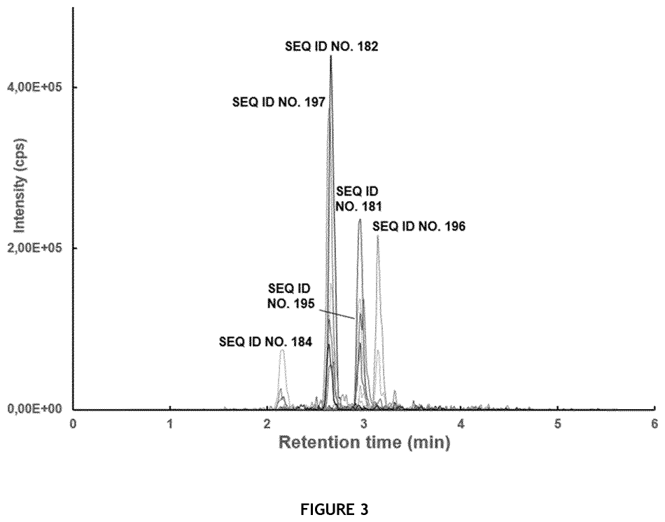

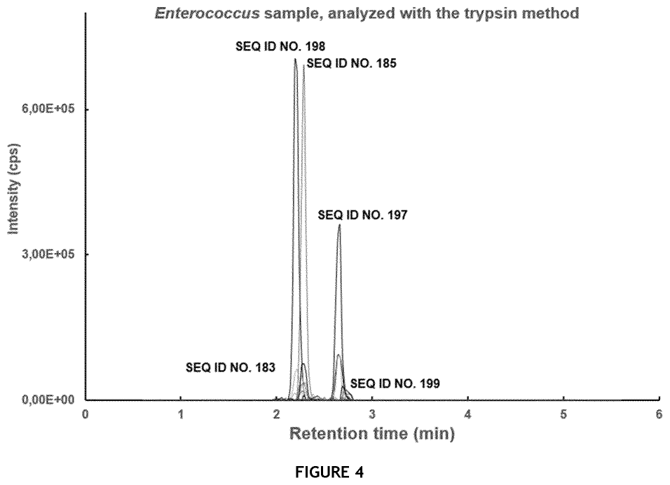

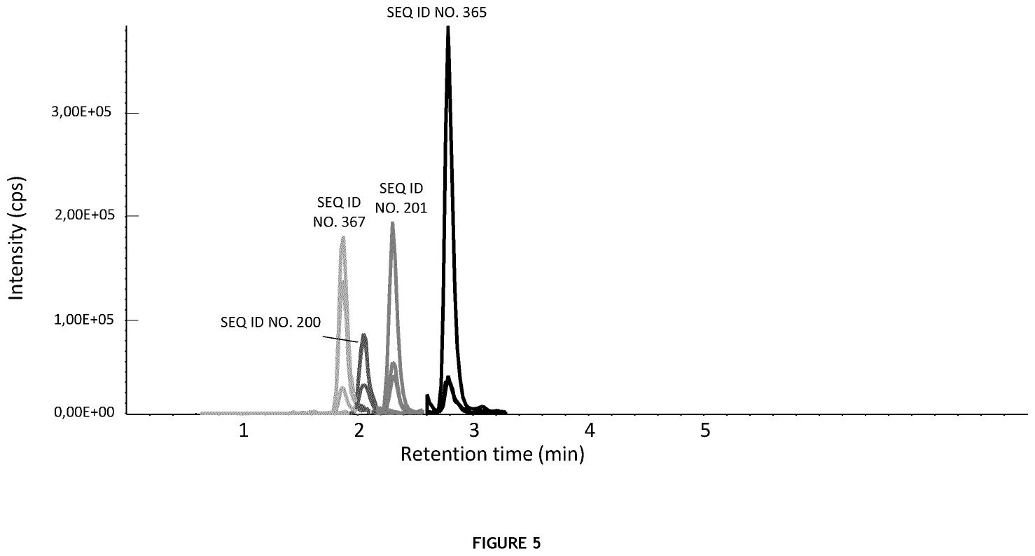

FIG. 1 shows a chromatogram obtained as an example of an endogenous Sentinel-MRM method. The intensity of the peptides are expressed in arbitrary units.

The interest of using two peptides as sentinel compounds, instead of only one, is to secure the trigger of a transition group. For example, if a peptide is mutated, or non-present in the analyzed sample, the second peptide will be able to trigger the transition group.

### Example 2: Construction of an Exogenous Sentinel-MRM Method with Exogenous Peptides (Trypsin) as Group Triggers

Trypsin is used as a digesting enzyme in the preparation of the samples. It is therefore present in excess in all samples; the peptides resulting from the self-digestion of trypsin itself can be used as sentinel compounds of groups of transitions. The principle is to distribute the specific peptides of each bacterial species (see table 1) into 4 groups triggered by 4 peptides resulting from the self-digestion of trypsin.

The FASTA sequence of trypsin (accession number P00761), hereafter referenced as SEQ ID NO. 428, is as follows:

FPTDDDDKIVGGYTCAANSIPYQVSLNSGSHFCGGSLINSQWVVSAAHCY
KSRIQVRLGEHNIDVLEGNEQFINAAKIITHPNFNGNTLDNDIMLIKLSS
PATLNSRVATVSLPRSCAAAGTECLISGWGNTKSSGSSYPSLLQCLKAPV
LSDSSCKSSYPGQITGNMICVGFLEGGKDSCQGDSGGPVVCNGQLQGIVS
WGYGCAQKNKPGVYTKVCNYVNWIQQTIAAN

The 4 peptides selected as Sentinel triggers (sentinel compounds) are as follows:

| (SEQ ID NO. 424) | |
|---|---|
| | NKPGVYTK |
| (SEQ ID NO. 425) | |
| | VATVSLPR |
| (SEQ ID NO. 426) | |
| | LGEHNIDVLEGNEQFINAAK |
| (SEQ ID NO. 427) | |
| | IITHPNFNGNTLDNDIMLIK |

FIG. 2 shows the distribution of these 4 peptides on a chromatogram.

One of the advantages of using peptides derived from trypsin is the possibility of following all the transitions of the selected peptides, without the risk of not triggering a group due to a low intensity (few quantity of bacteria present in the sample for example) or the triggering of a group by an interference in the sample.

Another advantage of this implementation is to be able to identify several bacteria in the context of a poly-infection, for example.

This implementation of the method could also be more easily incremented than the “endogenous” method with peptides from new or different species from the panel chosen in the context of this invention. For example, it will suffice to create an MRM method with the “new” peptides to know their retention time and to place them in the group of transitions corresponding to the order of elution of the tryptic peptides.

### Example 3: Identification of a Microorganism from a Positive Blood Culture by Sentinel-MRM Mode of Acquisition

1. Isolation of Microorganisms from a Positive Blood Culture

To isolate the bacteria/yeast present in a blood culture flask that has been detected as being positive, i.e., as comprising at least one microorganism, the procedure consists of lysing the blood cells using a lysis buffer (here, 12% SDS) and then recovering the bacteria by centrifugation.
Using a syringe and a 21G needle, take 1 mL of blood culture medium and transfer it to a 1.5 mL Eppendorf tube
Add 200 μL of 12% sodium dodecyl sulfate (SDS) then vortex for 10 seconds
Centrifuge for 2 minutes at 16100 g and remove the supernatant
Resuspend the pellet in 1 mL of physiological serum
Centrifuge for 1 minute at 16100 g and discard the supernatant
Resuspend the pellet in 1 mL of physiological serum

### 2. Generation of Peptides by Enzymatic Digestion and Cell Lysis

In a 1.5 mL Eppendorf LowBind tube add a spoon of glass beads (Glass beads, acid-washed, 150-212 μm, Sigma-Aldrich, ref G1145) for a height of about 3-4 mm (one third of the final volume)
Vortex the sample prepared according to paragraph 1 for 10 seconds then take 200 μl and dispense it in the tube containing the glass beads
Prepare a 1 mg/mL solution of trypsin in 150 mM Ammonium Bicarbonate buffer from lyophilized trypsin.
Add 50 μL of freshly prepared trypsin solution (1 mg/mL) to the tube containing the beads and bacteria/yeasts and vortex for 3 seconds
Place the sample in the water bath of the sonicator, for example a Diagenode, set at 50° C.
Immediately start the ultrasound 10 cycles of 1 minute
   30 seconds ultrasound ON
   30 seconds ultrasound OFF Ultrasonic power: Low
After digestion add 5 μL of formic acid and vortex for 3 seconds to stop the reaction
Centrifuge the tube at 9600 g (10,000 rpm on an Accuspin Micro 17 benchtop centrifuge) for 5 minutes
Transfer 150 μL of the supernatant into an amber vial fitted with an insert

### 3. Analytical Conditions: Chromatographic and Mass Spectrometric Conditions

Each sample is treated according to the protocols of paragraphs 1 and 2, then a volume of 5 μL of digested proteins is injected and analyzed under the following conditions:
HPLC device Agilent Pump 1290 of the company AGILENT (AGILENT, Santa Clara, United States of America)
WATERS chromatographic column (WATERS, Saint-Quentin en Yvelines, France) XBridge Peptide BEH C18, 1 mm internal diameter, 100 mm long, particle size 3.5 μm, pore size 130 Å)
Solvent A: H₂O 99.9%+0.1% formic acid
Solvent B: Acetonitrile 99.9%+0.1% formic acid
Column oven temperature: 60° C.
HPLC gradient defined according to table 2 defined below:

TABLE 2
chromatographic gradient

| | Time | Flow | Solvent | Solvent |
|---|---|---|---|---|
| Step | (min) | (μL/min) | A (%) | B (%) |
| Decomplexing | 0 | 100 | 98 | 2 |
| Decomplexing | 0.1 | 100 | 90 | 10 |
| Decomplexing | 4.22 | 100 | 65 | 35 |
| Washing | 4.25 | 100 | 25 | 75 |
| Washing | 4.28 | 200 | 25 | 75 |
| Washing | 4.30 | 300 | 25 | 75 |
| Washing | 5.22 | 300 | 25 | 75 |
| Equilibrating | 5.77 | 300 | 98 | 2 |
| Equilibrating | 5.78 | 200 | 98 | 2 |
| Equilibrating | 5.79 | 100 | 98 | 2 |
| Equilibrating | 7.00 | 100 | 98 | 2 |

The decomplexing step (i.e. partial separation) of the peptides is performed under conditions with less than 40% of solvent B, mainly composed of the polar solvent acetonitrile.

5 The eluate coming from the chromatographic column is injected directly into the ionization source of the QTRAP®6500+mass spectrometer from AB SCIEX (Framingham, Massachussetts, United States of America).

The other settings of the instrument are gathered in table 3 below.

TABLE 3
Settings of the mass spectrometer

| Scan type | Sentinel-MRM | |
|---|---|---|
| Polarity | Positive | |
| Ionisation source | Turbo Spray IonDrive (AB SCIEX) | |
| Resolution Q1 | unit | |
| Resolution Q3 | unit | |
| Dwell time | 5 | ms |
| Pause between mass ranges | 5 | ms |
| Scan rate | 10 | Da/sec |
| Curtain gas | 50.00 | psi |
| Ion spray voltage | 5500.00 | V |
| Source Temperature | 550.00° | C. |
| Gas source 1 (nebulising) | 70.00 | psi |
| Gas source 2 (drying) | 60.00 | psi |
| Collision Gas | high | |
| Entrance Potential (EP) | 10.00 | V |
| Collision Cell Exit Potential (CXP) | 12.00 | V |

| | |
|---|---|
| Software version | Analyst 1.7.2 |

The peptides resulting from the digestion of the proteins of the bacteria with trypsin are analyzed by the mass spectrometer in the Sentinel-MRM mode. The peptides tracked and detected allow the identification of the microorganism because they are specific to it.

4. Identification of a Microorganism by Sentinel-MRM Mode of Acquisition, Use of Endogenous Peptides such as Sentinel, Application to a Blood Culture Sample (Blind Identification)

The Sentinel-MRM method shown in Example 1 is applied to a blood culture sample.

The Sentinel-MRM acquisition method uses 3 or more transitions of each peptide (see the acquisition method in table 4), which constitutes a method containing more than 1500 transitions. To increase the specificity of the trigger Sentinel and thus avoid triggering a group by an interference present in the sample, it was decided that the alignment of three transitions was necessary to trigger a group.

The chromatogram obtained is shown in FIG. 3. The Enterococcus group was successfully triggered and only peptides specific to the genus Enterococcus and the species Enterococcus faecium were detected. This result was confirmed by MALDI-TOF.

5. Identification of the Microorganism by Sentinel-MRM Mode of Acquisition, Use of Trypsine-Issued Peptides as Sentinel Compounds The same sample as the example above is analyzed by applying the Sentinel-MRM acquisition method with trypsin peptides as Sentinel triggering groups of transitions.

Results are presented in FIG. 4. Only peptides specific to the genus Enterococcus and the species Enterococcus faecium were detected. This result was confirmed by MALDI-TOF.

### Example 4: Analysis Validation of the Two Acquisition Method in COMparison with the Analysis with a MALDI-TOF

To validate the two acquisition methods, 42 samples have been analyzed. The results are shown in table 4.

TABLE 4
Implementation of the method of the invention on 42 blood samples

| | | | Identification with | Identification with |
|---|---|---|---|---|
| | Flask | Identification | the « endogenous » | the « exogenous » |
| Blood | AEROBIC/ | with | method of | method of |
| sample | ANAEROBIC | MALDI-TOF | the invention | the invention |
| Sample1 | AER | Staphylococcus | Staphylococcus | Staphylococcus |
| | | epidermidis | coagulase negative | coagulase negative |
| Sample2 | AER | Escherichia coli | Escherichia coli | Escherichia coli |
| Sample3 | AER | Escherichia coli | Escherichia coli | Escherichia coli |
| Sample4 | ANA | Klebsiella pneumoniae | Klebsiella pneumoniae | Klebsiella pneumoniae |
| Sample5 | AER | Morganella morganii | Morganella morganii | Morganella morganii |
| Sample6 | AER | Klebsiella pneumoniae | Klebsiella pneumoniae | Klebsiella pneumoniae |
| Sample7 | ANA | Klebsiella aerogenes | Klebsiella aerogenes | Klebsiella aerogenes |
| Sample8 | AER | Klebsiella pneumoniae | Klebsiella pneumoniae | Klebsiella pneumoniae |
| Sample9 | AER | Escherichia coli | Escherichia coli | Escherichia coli |
| Sample10 | AER | Enterrococcus faecium | Enterrococcus faecium | Enterrococcus faecium |
| Sample11 | AER | Streptococcus | Streptococcus bovis | Streptococcus bovis |
| | | gallolyticus | and related | and related |
| Sample12 | AER | Klebsiella oxytoca | Klebsiella oxytoca | Klebsiella oxytoca |
| Sample13 | AER | Escherichia coli | Escherichia coli | Escherichia coli |
| Sample14 | AER | Staphylococcus aureus | Staphylococcus aureus | Staphylococcus aureus |
| | | | or argenteus | or argenteus |
| Sample15 | AER | Escherichia coli | Escherichia coli | Escherichia coli |
| Sample16 | AER | Staphylococcus aureus | Staphylococcus aureus | Staphylococcus aureus or |
| | | and Bacillus cereus | or argenteus | argenteus and Bacillus |
| Sample17 | AER | Enterrococcus faecalis | Enterrococcus faecalis | Enterrococcus faecalis |
| Sample18 | AER | Enterobacter cloacae | Enterobacter asburiae | Enterobacter asburiae |
| | | complex | hormachei cloacae | hormachei cloacae |
| Sample19 | ANA | Proteus mirabilis | Proteus mirabilis | Proteus mirabilis |
| Sample20 | AER | Enterobacter cloacae | Enterobacter asburiae | Enterobacter asburiae |
| | | | hormachei cloacae | hormachei cloacae |
| Sample21 | AER | Escherichia coli | Escherichia coli | Escherichia coli |
| Sample22 | AER | Klebsiella pneumoniae | Klebsiella pneumoniae | Klebsiella pneumoniae |
| Sample23 | AER | Escherichia coli | Escherichia coli | Escherichia coli |
| Sample24 | AER | Escherichia coli | Escherichia coli | Escherichia coli |
| Sample25 | AER | Escherichia coli and | Escherichia coli and | Escherichia coli and |
| | | Klebsiella pneumoniae | Klebsiella pneumoniae | Klebsiella pneumoniae |
| Sample26 | AER | Pseudomonas aeruginosa | Pseudomonas aeruginosa | Pseudomonas aeruginosa |
| Sample27 | AER | Escherichia coli | Escherichia coli | Escherichia coli |
| Sample28 | AER | Klebsiella pneumoniae | Klebsiella pneumoniae | Klebsiella pneumoniae |
| Sample29 | ANA | Escherichia coli | Escherichia coli | Escherichia coli |
| Sample30 | AER | Pseudomonas aeruginosa | Pseudomonas aeruginosa | Pseudomonas aeruginosa |
| Sample31 | AER | Enterrococcus faecalis | Enterrococcus faecalis | Enterrococcus faecalis |
| Sample32 | ANA | Bacillus spp | Bacillus cereus or | Bacillus cereus or |
| | | | Bacillus simplex | Bacillus simplex |
| Sample33 | AER | Staphylococcus | Staphylococcus | Staphylococcus |
| | | epidermidis | coagulase negative | coagulase negative |
| Sample34 | AER | Escherichia coli | Escherichia coli | Escherichia coli |
| Sample35 | AER | Staphylococcus | Staphylococcus | Staphylococcus |
| | | epidermidis | coagulase negative | coagulase negative |
| Sample36 | AER | Klebsiella pneumoniae | Klebsiella pneumoniae | Klebsiella pneumoniae |
| Sample37 | AER | Klebsiella aerogenes | Klebsiella aerogenes | Klebsiella aerogenes |
| Sample38 | AER | Pseudomonas aeruginosa | Pseudomonas aeruginosa | Pseudomonas aeruginosa |
| Sample39 | ANA | Escherichia coli | Escherichia coli | Escherichia coli |
| Sample40 | AER | Proteus mirabilis | Proteus mirabilis | Proteus mirabilis |
| Sample41 | AER | Staphylococcus | Staphylococcus | Staphylococcus |
| | | haemolyticus | coagulase negative | coagulase negative |
| Sample42 | AER | Staphylococcus | Staphylococcus | Staphylococcus |
| | | epidermidis | coagulase negative | coagulase negative |

Other tests on 264 positive blood culture (bacteria or yeast) samples obtained from patients have been conducted.

Results obtained with the process of the invention have been compared with those obtained with MALDI-TOF MS identification technique: a correlation of 100% of the results have been observed.

Regarding the percentage of identification, the process of the invention allowed the identification of at least one bacterial or yeast species in 93% of the assayed samples. In 7% of the samples, no identification could be obtained, probably due to an insufficient amount of microorganisms in the sample, or because the sample was actually microorganism-free.

### Example 5. Identification of Two Different Bacterial Species Present in a Sample, via the Trypsin-Method

A positive blood sample has been analyzed by applying the Sentinel-MRM acquisition method with trypsin peptides as Sentinel triggering groups of transitions.

Results are presented in FIG. 5. Peptides specific to the group Streptococcus bovis (SEQ ID NO: 365, 367) and the species Escherichia coli (SEQ ID NO. 200, 201) were detected. This result was confirmed by MALDI-TOF the day after, after an over-night sub-culturing step of the blood sample.

Advantageously, the process of the invention allows the identification of at least two different species in a same sample.

### REFERENCES CITED IN ORDER OF CITATION IN THE TEXT

### PATENTS

WO 2011/045544
WO 2012/143535
WO 2012/143534
EP 3384517
WO 2005/098071
WO 2014/116711

### BIBLIOGRAPHIC REFERENCES

Holland, R.D., Duffy, C.R., Rafii, F., Sutherland, J.B., Heinze, T.M., Holder, C.L., Voorhees, K.J., Lay, J.O., Jr., 1999. Identification of bacterial proteins observed in MALDI TOF mass spectra from whole cells. Analytical chemistry. 71, 3226-3230
Lasch P, Schneider A, Blumenscheit C, Doellinger J. Identification of Microorganisms by Liquid Chromatography-Mass Spectrometry (LC-MS1) and in Silico Peptide Mass Libraries. Mol Cell Proteomics. 2020 December; 19(12):2125-2139. doi: 10.1074/mcp.TIR120.002061. Epub 2020 Sep. 30. PMID: 32998977; PMCID: PMC7710138.
Boulund F, Karlsson R, Gonzales-Siles L, Johnning A, Karami N, Al-Bayati O, Åhrén C, Moore ERB, Kristiansson E. Typing and Characterization of Bacteria Using Bottom-up Tandem Mass Spectrometry Proteomics. Mol Cell Proteomics. 2017 June; 16(6):1052-1063.
Christian Blumenscheit, Yvonne Pfeifer, Guido Werner, Charlyn John, Andy Schneider, Peter Lasch, Joerg Doellinger. Unbiased antimicrobial resistance detection from clinical bacterial isolates using proteomics. bioRxiv 2020.11.17.386540; doi:

1. A method for the identification of at least one microorganism present in a sample based on the detection of peptides issued from the cleavage of ribosomal proteins of said microorganism, comprising the following steps:
a) lysis of microorganism(s) and cleavage of the proteins present in said sample to obtain a mixture of peptides,
b) decomplexing said peptides mixture using a liquid separation device coupled with a mass spectrometer,
c) nebulizing the liquid eluted from the separation device using an ion source in order to produce an ion current,
d) receiving said ion current from the ion source using said mass spectrometer and, for each cycle of a plurality of cycles, executing on the ion current a series of filtering steps for detecting a transition, said transition comprising a precursor ion and at least one fragment ion of said precursor ion, said transition being read from a predefined list of transitions using the mass spectrometer, wherein for each transition of the series, the mass spectrometer selects and fragments a precursor ion of the each transition,
e) receiving data concerning a plurality of transitions to be used to monitor the mixture of peptides using the processor,
f) assigning said plurality of transitions into two or more contiguous groups of transitions, into said predefined list of transitions using the processor,
g) monitoring at least one sentinel transition associated with one sentinel compound in each group of the two or more contiguous groups, wherein said at least one sentinel transition is selected as having the latest expected retention time in the group, using the processor,
h) when the signal of at least one sentinel transition of a group is detected with the mass spectrometer, starting the monitoring of at least one sentinel transition in a next contiguous group while stopping the monitoring of the transitions of the preceding group, using the processor,
i) optionally, generating a chromatogram or electropherogram, from the detection of transitions read from a predefined list with said mass spectrometer, using the processor,
wherein each transition read from the predefined listed is associated to a peptide, and
wherein the microorganism is identified according to the detection of said peptide(s).

2. The method according to claim 1, wherein the step of decomplexing the peptides mixture is performed by liquid chromatography or capillary electrophoresis.

3. The method according to claim 1, wherein the step of decomplexing the peptides mixture is carried out with a mobile phase comprising less than 40% of acetonitrile.

4. The method according to claim 1, wherein the mass spectrometer is a tandem mass spectrometer.

5. The method according to claim 1, wherein the mass spectrometer uses PRM (Parallel Reaction Monitoring), MRM (Multi Reaction Monitoring), DIA (Data Independent Acquisition), or SWATH MS (Sequential Window Acquisition of all THEoretical fragment ion spectra mass spectrometry).

6. The method according to claim 1, comprising a preliminary step of elimination of peptides that are not issued from the cleavage of ribosomal proteins by addition of a surfactant into said sample.

7. The method according to claim 1, wherein the two or more contiguous groups of transitions are associated with groups of peptides, each of the peptides being specific of a microorganism genus and/or species.

8. The method according to claim 1, wherein the cleavage of proteins is performed by digestion with the trypsin enzyme.

9. The method according to claim 1, wherein the predefined list of transitions comprises at least one transition that is associated to a peptide comprising between 6 and 20 amino-acids and that is decomplexed during step (b) with less than 40% of acetonitrile.

10. The method according to claim 1, wherein the predefined list of transitions comprises at least one transition that is associated to a peptide presenting a sequence selected from SEQ ID NO:1 to SEQ ID NO:423.

11. The method according to claim 1, wherein the sentinel compound is chosen from a group of compounds consisting of: peptides issued from step (a) of cleavage of proteins of the microorganism, peptides issued from autocleavage of the trypsin enzyme, peptides issued from the cleavage of proteins or peptides introduced into the sample, exogenous introduced peptides, and other compounds.

12. The method according to claim 1, wherein the sample is:
a biological sample obtained from a mammal, chosen from a group consisting of: blood, serum, lymph, mucus, stink, saliva, tracheobronchial aspirate, cerebrospinal fluid, and urine, or
a sample chosen from a group consisting of: used waters, food, drink, soil sample, and surface sample.

13. System for implementing the method according to claim 1, comprising a mass spectrometer coupled to a liquid separation device, and processing means configured for the implementation of the steps (e) to (h).

14. Group of peptides adapted for the implementation of the method according to claim 1, wherein said peptides are issued from ribosomal proteins, comprise 6 to 20 amino acids, and are decomplexed with a mobile phase comprising less than 40% of acetonitrile.

15. The group of peptides according to claim 14, comprising at least one peptide presenting a peptide sequence selected from SEQ ID NO:1 to SEQ ID NO:423.

16. The system according to claim 13, wherein the processing means are configured:
to receive data concerning a plurality of transitions to be used to monitor the mixture of peptides,
to assign the plurality of transitions into two or more contiguous groups of transitions into said predefined list of transitions,
to monitor at least one sentinel transition in each group of the two or more contiguous groups,
to start the monitoring of at least one transition in a next contiguous group, when the signal of at least one sentinel transition of a group is detected by the mass spectrometer, and
optionally, to generate a chromatogram or an electropherogram.


SEQUENCE LISTING (428 sequences)

SEQ ID NO: 1 - protein, 10 aa, Abiotrophia defectiva
SDEEAHALLK

SEQ ID NO: 2 - protein, 11 aa, Abiotrophia defectiva
GAMVLPHGTGK

SEQ ID NO: 3 - protein, 11 aa, Abiotrophia defectiva
GSEAVSLTVNR

SEQ ID NO: 4 - protein, 10 aa, Abiotrophia defectiva
FVDQMITLGK

SEQ ID NO: 5 - protein, 19 aa, Abiotrophia defectiva
AAALANLVEGSIVEGTVAR

SEQ ID NO: 6 - protein, 12 aa, Abiotrophia defectiva
EYAVVNLEALNR

SEQ ID NO: 7 - protein, 12 aa, Abiotrophia defectiva
LGFEGGQTQLFR

SEQ ID NO: 8 - protein, 10 aa, Abiotrophia defectiva
ELDLIGVGYR

SEQ ID NO: 9 - protein, 13 aa, Achromobacter xylosoxidans
LMQVILAPIVTEK

SEQ ID NO: 10 - protein, 12 aa, Achromobacter xylosoxidans
VVGALGQILGPR

SEQ ID NO: 11 - protein, 10 aa, Achromobacter xylosoxidans
VIEPLITLGK

SEQ ID NO: 12 - protein, 14 aa, Achromobacter xylosoxidans
GNTGETLIQLLESR

SEQ ID NO: 13 - protein, 14 aa, Acinetobacter baumannii
ILYEIEGVNEDLAR

SEQ ID NO: 14 - protein, 17 aa, Acinetobacter baumannii
TDLPEFAPGDTVVVQVK

SEQ ID NO: 15 - protein, 13 aa, Acinetobacter baumannii
ATIANVNASDEER

SEQ ID NO: 16 - protein, 12 aa, Acinetobacter baumannii
SGTTGNIEAATK

SEQ ID NO: 17 - protein, 10 aa, Acinetobacter baumannii
STGESVAVAK

SEQ ID NO: 18 - protein, 8 aa, Acinetobacter common
TLEQYFGR

SEQ ID NO: 19 - protein, 12 aa, Acinetobacter common
QGLGIAIVSTSK

SEQ ID NO: 20 - protein, 11 aa, Acinetobacter common
GGFTVDIGPVR

SEQ ID NO: 21 - protein, 11 aa, Acinetobacter common
GIQPVSPWGQK

SEQ ID NO: 22 - protein, 12 aa, Acinetobacter common
VEGDIVSLETLK

SEQ ID NO: 23 - protein, 15 aa, Acinetobacter common
AGDAAPMAYVELVDR

SEQ ID NO: 24 - protein, 8 aa, Acinetobacter common
AALDYGLK

SEQ ID NO: 25 - protein, 7 aa, Acinetobacter common
EISMNIK

SEQ ID NO: 26 - protein, 12 aa, Acinetobacter common
EPDLTGADLDAR

SEQ ID NO: 27 - protein, 7 aa, Acinetobacter common
NVMEIPR

SEQ ID NO: 28 - protein, 10 aa, Acinetobacter common
LADEVEATLK

SEQ ID NO: 29 - protein, 11 aa, Acinetobacter common
FNVLTSPHVNK

SEQ ID NO: 30 - protein, 9 aa, Acinetobacter common
VNIASIQVK

SEQ ID NO: 31 - protein, 10 aa, Acinetobacter common
LIDIVQPTDK

SEQ ID NO: 32 - protein, 11 aa, Acinetobacter common
AFTVQGVALTK

SEQ ID NO: 33 - protein, 15 aa, Acinetobacter common
IFEDGEIVTGVISGK

SEQ ID NO: 34 - protein, 17 aa, Acinetobacter Iwoffii
GMAMNPVDHPHGGGEGR

SEQ ID NO: 35 - protein, 12 aa, Acinetobacter Iwoffii
AVEQLFGVEVVK

SEQ ID NO: 36 - protein, 13 aa, Acinetobacter Iwoffii
QLGEDPWLAIMNR

SEQ ID NO: 37 - protein, 13 aa, Acinetobacter Iwoffii
SIAESIVYGALDR

SEQ ID NO: 38 - protein, 7 aa, Acinetobacter pittii
LQLAPVK

SEQ ID NO: 39 - protein, 14 aa, Acinetobacter pittii
ILYEIEGVNEELAR

SEQ ID NO: 40 - protein, 7 aa, Acinetobacter pittii
LFEDFAK

SEQ ID NO: 41 - protein, 14 aa, Acinetobacter pittii
AQVLGDTVGVQVFK

SEQ ID NO: 42 - protein, 11 aa, Acinetobacter pittii
QPLELLEVTEK

SEQ ID NO: 43 - protein, 12 aa, Actinomyces odontolyticus
GTHFHPGDGVGR

SEQ ID NO: 44 - protein, 11 aa, Actinomyces odontolyticus
IQVFQGVVIAR

SEQ ID NO: 45 - protein, 16 aa, Actinomyces odontolyticus
TAGLTGENLVELLEMR

SEQ ID NO: 46 - protein, 17 aa, Actinomyces odontolyticus
VEDGIEGLVHISELAQR

SEQ ID NO: 47 - protein, 11 aa, Aerococcus viridans
EATASAVSAQR

SEQ ID NO: 48 - protein, 8 aa, Aerococcus viridans
GASSGWGK

SEQ ID NO: 49 - protein, 8 aa, Aerococcus viridans
MLDQAASK

SEQ ID NO: 50 - protein, 8 aa, Aerococcus viridans
IAIQEAHK

SEQ ID NO: 51 - protein, 11 aa, Aerococcus viridans
VGDTLELVVIK

SEQ ID NO: 52 - protein, 11 aa, Aerococcus viridans
YALSEAIELLK

SEQ ID NO: 53 - protein, 14 aa, Aerococcus viridans
NWVVLDATDVPLGR

SEQ ID NO: 54 - protein, 10 aa, Bacillus simplex
VATIEYDPNR

SEQ ID NO: 55 - protein, 10 aa, Bacillus simplex
MYAIIETGGK

SEQ ID NO: 56 - protein, 12 aa, Bacillus simplex
LDLPSGVDIEIK

SEQ ID NO: 57 - protein, 14 aa, Bacillus simplex
WLGGTLTNFETIQK

SEQ ID NO: 58 - protein, 12 aa, Bacillus simplex
EQLIFPEIDYDK

SEQ ID NO: 59 - protein, 9 aa, Bacillus simplex
MADAILEAK

SEQ ID NO: 60 - protein, 10 aa, Bacillus simplex
TGTVTFDVTK

SEQ ID NO: 61 - protein, 14 aa, Bacteroides fragilis
GITGEVLLQMLEGR

SEQ ID NO: 62 - protein, 10 aa, Bacteroides fragilis
LLVVLPEANK

SEQ ID NO: 63 - protein, 19 aa, Bacteroides fragilis
QLTPHPWDALDPNLQVGDK

SEQ ID NO: 64 - protein, 12 aa, Bacteroides fragilis
AFAEQLVNLTVK

SEQ ID NO: 65 - protein, 12 aa, Bacteroides fragilis
LNVVILDFDDEK

SEQ ID NO: 66 - protein, 14 aa, Bacteroides fragilis
VINGLGIAIISTSK

SEQ ID NO: 67 - protein, 7 aa, Bacteroides thetaiotaomicron
VGEMIAK

SEQ ID NO: 68 - protein, 14 aa, Bacteroides thetaiotaomicron
FIPVYVTENMVGHK

SEQ ID NO: 69 - protein, 14 aa, Bacteroides thetaiotaomicron
GAPEGFVAPVTPGR

SEQ ID NO: 70 - protein, 8 aa, Bacteroides thetaiotaomicron
TSFDVVLK

SEQ ID NO: 71 - protein, 10 aa, Bacteroides vulgatus
AGDTITVAYR

SEQ ID NO: 72 - protein, 9 aa, Bacteroides vulgatus
ALYNVIPER

SEQ ID NO: 73 - protein, 9 aa, Campylobacter coli
HSGYFGSVK

SEQ ID NO: 74 - protein, 10 aa, Campylobacter coli
LDVGDALLVR

SEQ ID NO: 75 - protein, 12 aa, Campylobacter coli
SLGSNNSANVVR

SEQ ID NO: 76 - protein, 8 aa, Campylobacter coli
FMYGVSEK

SEQ ID NO: 77 - protein, 12 aa, Campylobacter coli
LAAELLDAANSK

SEQ ID NO: 78 - protein, 9 aa, Campylobacter coli
ALMDLGSFR

SEQ ID NO: 79 - protein, 17 aa, Campylobacter coli
MLDIVAATPDTVDSLTK

SEQ ID NO: 80 - protein, 11 aa, Campylobacter coli
LLELIGVPFTK

SEQ ID NO: 81 - protein, 15 aa, Candida albicans
VETGNFSWGSEGVSR

SEQ ID NO: 82 - protein, 13 aa, Candida albicans
IIVAPIATETAMK

SEQ ID NO: 83 - protein, 12 aa, Candida albicans
YGNVNNDFVLLK

SEQ ID NO: 84 - protein, 11 aa, Candida albicans
QVVFEIPGESH

SEQ ID NO: 85 - protein, 11 aa, Candida albicans
SVDAALLSEIK

SEQ ID NO: 86 - protein, 11 aa, Candida albicans
AVASGASVVSK

SEQ ID NO: 87 - protein, 7 aa, Candida auris
APSTFER

SEQ ID NO: 88 - protein, 7 aa, Candida auris
AVEVPEK

SEQ ID NO: 89 - protein, 7 aa, Candida auris
TAYETLR

SEQ ID NO: 90 - protein, 8 aa, Candida auris
LVMVTGGK

SEQ ID NO: 91 - protein, 8 aa, Candida auris
VGVLPEDK

SEQ ID NO: 92 - protein, 7 aa, Candida auris
AVDPFAK

SEQ ID NO: 93 - protein, 7 aa, Candida auris
EVGLGFK

SEQ ID NO: 94 - protein, 9 aa, Candida auris
VAPLPLAAK

SEQ ID NO: 95 - protein, 8 aa, Candida auris
LLAGLPIR

SEQ ID NO: 96 - protein, 11 aa, Candida auris
SPLDVFSEEAK

SEQ ID NO: 97 - protein, 14 aa, Candida auris
SIVSEVSGLAPYER

SEQ ID NO: 98 - protein, 10 aa, Candida auris
TINPLGGFVR

SEQ ID NO: 99 - protein, 12 aa, Candida auris
VIDLQAPAQIVK

SEQ ID NO: 100 - protein, 11 aa, Candida auris
GQLPQVPIIVK

SEQ ID NO: 101 - protein, 15 aa, Candida auris
ALAIFVPVPSLVGYR

SEQ ID NO: 102 - protein, 12 aa, Candida common
TSFFQALGVPTK

SEQ ID NO: 103 - protein, 8 aa, Candida common
AFLIEEQK

SEQ ID NO: 104 - protein, 7 aa, Candida common
LLGTAFK

SEQ ID NO: 105 - protein, 9 aa, Candida common
IGPLGLSPK

SEQ ID NO: 106 - protein, 7 aa, Candida common
FQTPAEK

SEQ ID NO: 107 - protein, 7 aa, Candida common
TFGASVR

SEQ ID NO: 108 - protein, 9 aa, Candida glabrata
ALSEQAEAR

SEQ ID NO: 109 - protein, 7 aa, Candida glabrata
TLVQAPR

SEQ ID NO: 110 - protein, 7 aa, Candida glabrata
TPVTLAR

SEQ ID NO: 111 - protein, 8 aa, Candida glabrata
DDEVLVTR

SEQ ID NO: 112 - protein, 10 aa, Candida glabrata
LAASVIGAGK

SEQ ID NO: 113 - protein, 8 aa, Candida glabrata
VISDILTR

SEQ ID NO: 114 - protein, 7 aa, Candida glabrata
QFLELTR

SEQ ID NO: 115 - protein, 11 aa, Candida glabrata
GVIGVIAGGGR

SEQ ID NO: 116 - protein, 9 aa, Candida glabrata
YTLDVESFK

SEQ ID NO: 117 - protein, 11 aa, Candida glabrata
LLEMSTEDFIK

SEQ ID NO: 118 - protein, 8 aa, Candida glabrata
GFSLAEIK

SEQ ID NO: 119 - protein, 10 aa, Candida glabrata
QIVFEIPETH

SEQ ID NO: 120 - protein, 7 aa, Candida kefyr
QYATVSR

SEQ ID NO: 121 - protein, 7 aa, Candida kefyr
TPGGVLR

SEQ ID NO: 122 - protein, 13 aa, Candida kefyr
VIEQPITSETAMK

SEQ ID NO: 123 - protein, 9 aa, Candida kefyr
VVYALTTIR

SEQ ID NO: 124 - protein, 11 aa, Candida kefyr
AVVGASLELIK

SEQ ID NO: 125 - protein, 9 aa, Candida kefyr
LWTLVPEEK

SEQ ID NO: 126 - protein, 12 aa, Candida kefyr
EGDILVLMESER

SEQ ID NO: 127 - protein, 11 aa, Candida krusei
AVVVPEQTAYR

SEQ ID NO: 128 - protein, 8 aa, Candida krusei
ALEQVNLK

SEQ ID NO: 129 - protein, 13 aa, Candida krusei
VIQSPITSESATK

SEQ ID NO: 130 - protein, 9 aa, Candida krusei
VFLDVGLQR

SEQ ID NO: 131 - protein, 10 aa, Candida krusei
TITPMGGFVR

SEQ ID NO: 132 - protein, 11 aa, Candida krusei
AIVGASLDLIK

SEQ ID NO: 133 - protein, 14 aa, Candida krusei
ILDDLVFPTEIVGK

SEQ ID NO: 134 - protein, 12 aa, Candida krusei
FTPGSFTNYITK

SEQ ID NO: 135 - protein, 12 aa, Candida krusei
SAIVQIDATPFK

SEQ ID NO: 136 - protein, 12 aa, Candida krusei
MIIIAANTPVLR

SEQ ID NO: 137 - protein, 10 aa, Candida parapsilosis
NSLVHDGLAR

SEQ ID NO: 138 - protein, 11 aa, Candida parapsilosis
AVEVPEQSAYR

SEQ ID NO: 139 - protein, 7 aa, Candida parapsilosis
LLVQQPR

SEQ ID NO: 140 - protein, 15 aa, Candida parapsilosis
IAGVVYHPSNNELVR

SEQ ID NO: 141 - protein, 12 aa, Candida parapsilosis
LISTIDANYLQK

SEQ ID NO: 142 - protein, 12 aa, Candida parapsilosis
ILAESPSPLDLK

SEQ ID NO: 143 - protein, 11 aa, Candida parapsilosis
QVVFEIPGETH

SEQ ID NO: 144 - protein, 15 aa, Candida parapsilosis
ALAIFVPPPSVVGYR

SEQ ID NO: 145 - protein, 7 aa, Candida tropicalis
YASSIGR

SEQ ID NO: 146 - protein, 11 aa, Candida tropicalis
LAASGASVVSK

SEQ ID NO: 147 - protein, 11 aa, Candida tropicalis
NFGIGQSVQPK

SEQ ID NO: 148 - protein, 11 aa, Candida tropicalis
QVVFEIPGENH

SEQ ID NO: 149 - protein, 17 aa, Candida tropicalis
SGYTLPANIISNTDVTR

SEQ ID NO: 150 - protein, 15 aa, Candida tropicalis
ALAVFVPPPSLAAYR

SEQ ID NO: 151 - protein, 13 aa, Capnocytophaga sputigena
TAPAAVQLLEAAK

SEQ ID NO: 152 - protein, 12 aa, Capnocytophaga sputigena
NFAEQLVNLTVK

SEQ ID NO: 153 - protein, 13 aa, Capnocytophaga sputigena
STLGDLEVLQELK

SEQ ID NO: 154 - protein, 14 aa, Capnocytophaga sputigena
IMFEVGGVPLDVAK

SEQ ID NO: 155 - protein, 14 aa, Citrobacter freundii and Citrobacter braakii
GNTGENLLGLLEGR

SEQ ID NO: 156 - protein, 11 aa, Citrobacter freundii and Citrobacter braakii
LADVLSAAEAR

SEQ ID NO: 157 - protein, 8 aa, Citrobacter koseri
LATELALR

SEQ ID NO: 158 - protein, 10 aa, Citrobacter koseri
VSVVNNPTGR

SEQ ID NO: 159 - protein, 15 aa, Clostridium perfringens
NALYTPAEALELAVK

SEQ ID NO: 160 - protein, 16 aa, Clostridium perfringens
ALLNNMVVGVSQGFSK

SEQ ID NO: 161 - protein, 12 aa, Clostridium perfringens
VLFELSGVDEEK

SEQ ID NO: 162 - protein, 12 aa, Clostridium perfringens
EIETYFGLETLR

SEQ ID NO: 163 - protein, 13 aa, Clostridium perfringens
AIVNILLQEGYLK

SEQ ID NO: 164 - protein, 12 aa, Eggerthella lenta
EALVNYALTPFK

SEQ ID NO: 165 - protein, 9 aa, Eggerthella lenta
SLLAPLSNK

SEQ ID NO: 166 - protein, 10 aa, Eggerthella lenta
LLDAAMGDLR

SEQ ID NO: 167 - protein, 8 aa, Eggerthella lenta
AADLLVIK

SEQ ID NO: 168 - protein, 9 aa, Enterobacter asburiae
GISNVSFDR

SEQ ID NO: 169 - protein, 10 aa, Enterobacterales common
YLSLLPYTDR

SEQ ID NO: 170 - protein, 12 aa, Enterobacterales common
QLGEDPWVAIAK

SEQ ID NO: 171 - protein, 11 aa, Enterobacterales common
FTVLISPHVNK

SEQ ID NO: 172 - protein, 10 aa, Enterobacterales common
VVEPLITLAK

SEQ ID NO: 173 - protein, 12 aa, Enterobacterales common
LVADSITSQLER

SEQ ID NO: 174 - protein, 6 aa, Enterobacterales common
FGFTSR

SEQ ID NO: 175 - protein, 14 aa, Enterobacterales common
VANLGSLGDQVNVK

SEQ ID NO: 176 - protein, 11 aa, Enterobacterales common
GGFTVELNGIR

SEQ ID NO: 177 - protein, 8 aa, Enterobacterales common
NYITESGK

SEQ ID NO: 178 - protein, 11 aa, Enterobacterales common
AVIESENSAER

SEQ ID NO: 179 - protein, 8 aa, Enterobacterales common
NMAGSLVR

SEQ ID NO: 180 - protein, 10 aa, Enterobacterales common
FVNILMVDGK

SEQ ID NO: 181 - protein, 10 aa, Enterococcus common
EGVVLAAFPK

SEQ ID NO: 182 - protein, 9 aa, Enterococcus common
TQTVLVFAK

SEQ ID NO: 183 - protein, 10 aa, Enterococcus common
LVDAAYDYMK

SEQ ID NO: 184 - protein, 11 aa, Enterococcus common
NVELGEYEVGK

SEQ ID NO: 185 - protein, 12 aa, Enterococcus common
SLGSNTPINVVR

SEQ ID NO: 186 - protein, 10 aa, Enterococcus common
FLGGIADMPR

SEQ ID NO: 187 - protein, 10 aa, Enterococcus common
SVADAISILK

SEQ ID NO: 188 - protein, 14 aa, Enterococcus faecalis
EEDESIVVESALQK

SEQ ID NO: 189 - protein, 11 aa, Enterococcus faecalis
QVLANLSIDTK

SEQ ID NO: 190 - protein, 11 aa, Enterococcus faecalis
VVVLPAGVEIK

SEQ ID NO: 191 - protein, 12 aa, Enterococcus faecalis
VSSVEQITALAK

SEQ ID NO: 192 - protein, 13 aa, Enterococcus faecalis
LADAAVSTIEIER

SEQ ID NO: 193 - protein, 13 aa, Enterococcus faecalis
EVINQPFGVTETK

SEQ ID NO: 194 - protein, 13 aa, Enterococcus faecalis
FEDGTEVTPVVLK

SEQ ID NO: 195 - protein, 12 aa, Enterococcus faecium
VYPVAEAVALAK

SEQ ID NO: 196 - protein, 12 aa, Enterococcus faecium
VSSLEEITALAK

SEQ ID NO: 197 - protein, 10 aa, Enterococcus faecium
VTIQNLEVVR

SEQ ID NO: 198 - protein, 9 aa, Enterococcus faecium
NVQPVLEVK

SEQ ID NO: 199 - protein, 14 aa, Enterococcus faecium
EENEDIVIESALQK

SEQ ID NO: 200 - protein, 11 aa, Escherichia coli
YTAAITGAEGK

SEQ ID NO: 201 - protein, 7 aa, Escherichia coli
YTQLIER

SEQ ID NO: 202 - protein, 8 aa, Fusobacterium necrophorum
LVNDELDK

SEQ ID NO: 203 - protein, 8 aa, Fusobacterium necrophorum
SEWAVEGK

SEQ ID NO: 204 - protein, 9 aa, Fusobacterium necrophorum
AGMYYVNSR

SEQ ID NO: 205 - protein, 10 aa, Fusobacterium necrophorum
EMTSEDLVVK

SEQ ID NO: 206 - protein, 9 aa, Fusobacterium necrophorum
DYNLYLSAR

SEQ ID NO: 207 - protein, 7 aa, Fusobacterium necrophorum
NAFAFLR

SEQ ID NO: 208 - protein, 13 aa, Fusobacterium necrophorum
FQLSLGQLTNTAK

SEQ ID NO: 209 - protein, 9 aa, Granulicatella adiacens
ELTNDELDR

SEQ ID NO: 210 - protein, 13 aa, Granulicatella adiacens
NVAVTTTFGPGVK

SEQ ID NO: 211 - protein, 14 aa, Granulicatella adiacens
AVVELAGISDVTSK

SEQ ID NO: 212 - protein, 17 aa, Granulicatella adiacens
IGNKPVVIPAGVTVDLK

SEQ ID NO: 213 - protein, 12 aa, Granulicatella adiacens
AYPVQEAIALAK

SEQ ID NO: 214 - protein, 12 aa, Granulicatella adiacens
EAGLEGMDDVFK

SEQ ID NO: 215 - protein, 13 aa, Granulicatella adiacens
AATILYNAFDIVK

SEQ ID NO: 216 - protein, 10 aa, Granulicatella adiacens
ELELIGVGYR

SEQ ID NO: 217 - protein, 8 aa, Granulicatella adiacens
VAIANILK

SEQ ID NO: 218 - protein, 14 aa, Haemophilus influenzae
VNHWVAQGASLSDR

SEQ ID NO: 219 - protein, 12 aa, Haemophilus influenzae
DVAEAVTAAGVK

SEQ ID NO: 220 - protein, 12 aa, Haemophilus influenzae
SAAEAAFVEMQK

SEQ ID NO: 221 - protein, 12 aa, Haemophilus influenzae
ENLQALLAALNK

SEQ ID NO: 222 - protein, 12 aa, Haemophilus influenzae
AYEINEAIAVLK

SEQ ID NO: 223 - protein, 13 aa, Hafnia alvei
VSQALETLAYTNK

SEQ ID NO: 224 - protein, 10 aa, Hafnia alvei
GIETVLAEIR

SEQ ID NO: 225 - protein, 15 aa, Hafnia alvei
DVTGIDPVSLIAFDK

SEQ ID NO: 226 - protein, 14 aa, Hafnia alvei
SVEELNTELLSLLR

SEQ ID NO: 227 - protein, 10 aa, Hafnia alvei
ATIDGLASMK

SEQ ID NO: 228 - protein, 9 aa, Hafnia alvei
MFTIEATAR

SEQ ID NO: 229 - protein, 10 aa, Hafnia alvei
IGVPFVSGGK

SEQ ID NO: 230 - protein, 9 aa, Klebsiella aerogenes
AQIVSEFGR

SEQ ID NO: 231 - protein, 15 aa, Klebsiella aerogenes
DIADAVSAAGVAVAK

SEQ ID NO: 232 - protein, 15 aa, Klebsiella oxytoca
AYEDAETVVGIINGK

SEQ ID NO: 233 - protein, 18 aa, Klebsiella oxytoca
LSDLAHVEGDVIDLNTLK

SEQ ID NO: 234 - protein, 16 aa, Klebsiella oxytoca
VGFFNPIASATEEGTR

SEQ ID NO: 235 - protein, 18 aa, Klebsiella pneumoniae
LSDLAHVEGDVVDLNALK

SEQ ID NO: 236 - protein, 8 aa, Lactobacillus casei
VANVDMNR

SEQ ID NO: 237 - protein, 19 aa, Lactobacillus casei
AAAPVAAGAAAGGDAAATK

SEQ ID NO: 238 - protein, 9 aa, Lactobacillus casei
VPAGVTVTK

SEQ ID NO: 239 - protein, 12 aa, Lactobacillus casei
TLLVSQSHTAGR

SEQ ID NO: 240 - protein, 10 aa, Lactobacillus casei
HESLVVPASK

SEQ ID NO: 241 - protein, 10 aa, Lactobacillus casei
AYVNEGPTLK

SEQ ID NO: 242 - protein, 12 aa, Lactobacillus casei
DASILELNDLVK

SEQ ID NO: 243 - protein, 10 aa, Lactobacillus casei
LSSVVASILR

SEQ ID NO: 244 - protein, 12 aa, Lactobacillus casei
LNEEDILNWLQK

SEQ ID NO: 245 - protein, 9 aa, Morganella morganii
NIHNAVEVK

SEQ ID NO: 246 - protein, 9 aa, Morganella morganii
HTGYVGGIK

SEQ ID NO: 247 - protein, 8 aa, Morganella morganii
VSEGQIVR

SEQ ID NO: 248 - protein, 8 aa, Morganella morganii
ANLAAQIK

SEQ ID NO: 249 - protein, 16 aa, Morganella morganii
VGTVTPNVAEAVNNAK

SEQ ID NO: 250 - protein, 13 aa, Morganella morganii
AANVIGPQIEYAK

SEQ ID NO: 251 - protein, 8 aa, Morganella morganii
SLENYFGR

SEQ ID NO: 252 - protein, 9 aa, Morganella morganii
VFLSGELTR

SEQ ID NO: 253 - protein, 10 aa, Morganella morganii
VEGVNTLLVK

SEQ ID NO: 254 - protein, 11 aa, Morganella morganii
LYLGAVATSVR

SEQ ID NO: 255 - protein, 11 aa, Morganella morganii
IGFFNPIAAGK

SEQ ID NO: 256 - protein, 14 aa, Morganella morganii
TVPMFNDALAELNK

SEQ ID NO: 257 - protein, 16 aa, Morganella morganii
ALVNAMVVGVTEGFSK

SEQ ID NO: 258 - protein, 12 aa, Morganella morganii
LYDINEAVALLK

SEQ ID NO: 259 - protein, 14 aa, Morganella morganii
GNTGENLLSLLEGR

SEQ ID NO: 260 - protein, 15 aa, Morganella morganii
DVTAIDPVSLIAFGK

SEQ ID NO: 261 - protein, 11 aa, Morganella morganii
ALLSAFNFPFR

SEQ ID NO: 262 - protein, 8 aa, Morganella morganii
GTVVAIDK

SEQ ID NO: 263 - protein, 11 aa, Morganella morganii
GATVLPNGTGR

SEQ ID NO: 264 - protein, 8 aa, Morganella morganii
AGNALPMR

SEQ ID NO: 265 - protein, 13 aa, Neisseria meningitidis
SGSNVEAAAIVGK

SEQ ID NO: 266 - protein, 17 aa, Neisseria meningitidis
MFLNTIQPAVGATHAGR

SEQ ID NO: 267 - protein, 11 aa, Neisseria meningitidis
GLIEFALTEEK

SEQ ID NO: 268 - protein, 18 aa, Neisseria meningitidis
DLQVLMGVPVHVNIEEIR

SEQ ID NO: 269 - protein, 12 aa, Neisseria meningitidis
AGMATILSQLTR

SEQ ID NO: 270 - protein, 10 aa, Neisseria meningitidis
SWVVSELVEK

SEQ ID NO: 271 - protein, 8 aa, Ochrobactrum anthropi
TAFIALIR

SEQ ID NO: 272 - protein, 12 aa, Ochrobactrum anthropi
VLDVLQSEGYIR

SEQ ID NO: 273 - protein, 8 aa, Ochrobactrum anthropi
FITIALPR

SEQ ID NO: 274 - protein, 12 aa, Pantoea agglomerans
APVVVPAGVEVK

SEQ ID NO: 275 - protein, 8 aa, Pantoea agglomerans
EGSYVTLR

SEQ ID NO: 276 - protein, 14 aa, Pantoea agglomerans
GLPTPVVITVYSDR

SEQ ID NO: 277 - protein, 11 aa, Pantoea agglomerans
QPLELLDLVGK

SEQ ID NO: 278 - protein, 13 aa, Parvimonas micra
ILDALTIDAFSTK

SEQ ID NO: 279 - protein, 13 aa, Parvimonas micra
ITGDALVMMLETR

SEQ ID NO: 280 - protein, 14 aa, Parvimonas micra
VLFEMSGVPVDVAR

SEQ ID NO: 281 - protein, 11 aa, Parvimonas micra
ALLELLGMPFK

SEQ ID NO: 282 - protein, 12 aa, Proteus mirabilis
QYEINEAVALLK

SEQ ID NO: 283 - protein, 9 aa, Proteus mirabilis
EAFQLAAAK

SEQ ID NO: 284 - protein, 9 aa, Proteus mirabilis
VDFNEAQLK

SEQ ID NO: 285 - protein, 7 aa, Proteus mirabilis
IVAEFGR

SEQ ID NO: 286 - protein, 9 aa, Proteus mirabilis
ATMAGLGLR

SEQ ID NO: 287 - protein, 13 aa, Proteus vulgaris
LATLPTYDEAIAR

SEQ ID NO: 288 - protein, 12 aa, Proteus vulgaris
QYEITEAVALLK

SEQ ID NO: 289 - protein, 9 aa, Proteus vulgaris
VDFNETQLK

SEQ ID NO: 290 - protein, 11 aa, Pseudomonas aeruginosa
AALSAVVADAR

SEQ ID NO: 291 - protein, 12 aa, Pseudomonas aeruginosa
IIQQIEAEQMNK

SEQ ID NO: 292 - protein, 8 aa, Pseudomonas aeruginosa
LPAGVEIK

SEQ ID NO: 293 - protein, 15 aa, Pseudomonas aeruginosa
VHPSVEVIQDSGELR

SEQ ID NO: 294 - protein, 7 aa, Pseudomonas aeruginosa
YTALIGR

SEQ ID NO: 295 - protein, 9 aa, Pseudomonas aeruginosa
STPAAVLLK

SEQ ID NO: 296 - protein, 12 aa, Pseudomonas aeruginosa
VEGDVVSLQTLK

SEQ ID NO: 297 - protein, 10 aa, Pseudomonas aeruginosa
IGLPVVEGAK

SEQ ID NO: 298 - protein, 10 aa, Pseudomonas aeruginosa
VTVQSLEIVR

SEQ ID NO: 299 - protein, 10 aa, Pseudomonas aeruginosa
LVVGGVNLIK

SEQ ID NO: 300 - protein, 8 aa, Pseudomonas aeruginosa
AGDVVAVR

SEQ ID NO: 301 - protein, 9 aa, Pseudomonas oryzihabitans
TVLNQQAGK

SEQ ID NO: 302 - protein, 8 aa, Pseudomonas oryzihabitans
STGEVHLR

SEQ ID NO: 303 - protein, 11 aa, Pseudomonas oryzihabitans
SVLEAENSAER

SEQ ID NO: 304 - protein, 12 aa, Pseudomonas oryzihabitans
ALDAIAPLVEVK

SEQ ID NO: 305 - protein, 7 aa, Pseudomonas oryzihabitans
FGFVSLK

SEQ ID NO: 306 - protein, 16 aa, Raoultella ornithinolytica
ALLNSVVIGVTEGFTK

SEQ ID NO: 307 - protein, 8 aa, Raoultella ornithinolytica
SATGLGLK

SEQ ID NO: 308 - protein, 12 aa, Rothia dentocariosa
QAQEVIAEQATR

SEQ ID NO: 309 - protein, 15 aa, Rothia dentocariosa
AHETLAATGVNPDTR

SEQ ID NO: 310 - protein, 8 aa, Rothia dentocariosa
HIMVDGVR

SEQ ID NO: 311 - protein, 11 aa, Rothia dentocariosa
IVYGALEGVEK

SEQ ID NO: 312 - protein, 13 aa, Rothia dentocariosa
AEAAEEPMAAWER

SEQ ID NO: 313 - protein, 10 aa, Rothia dentocariosa
LIDVVDPTPK

SEQ ID NO: 314 - protein, 8 aa, Rothia dentocariosa
IIDIDMDR

SEQ ID NO: 315 - protein, 10 aa, Rothia dentocariosa
VLNLNESVMR

SEQ ID NO: 316 - protein, 12 aa, Rothia dentocariosa
ELSLVEVSEFVK

SEQ ID NO: 317 - protein, 18 aa, Rothia dentocariosa
TADPVTVGMVNTVAHLVK

SEQ ID NO: 318 - protein, 11 aa, Rothia dentocariosa
LSIEELIAAFK

SEQ ID NO: 319 - protein, 12 aa, Rothia dentocariosa
GSVILPEAITPK

SEQ ID NO: 320 - protein, 15 aa, Salmonella enterica
DIADAVTAAGVDVAK

SEQ ID NO: 321 - protein, 14 aa, Salmonella enterica
VAVFTQGPNAEAAK

SEQ ID NO: 322 - protein, 8 aa, Serratia marcescens
SLDTDGYR

SEQ ID NO: 323 - protein, 9 aa, Serratia marcescens
TIHDAVEVK

SEQ ID NO: 324 - protein, 12 aa, Serratia marcescens
INELGSVTIASK

SEQ ID NO: 325 - protein, 11 aa, Serratia marcescens
QEANALTFAPR

SEQ ID NO: 326 - protein, 15 aa, Serratia marcescens
DVAGIDPVSLIAFDK

SEQ ID NO: 327 - protein, 8 aa, Serratia marcescens
DGSYVTLR

SEQ ID NO: 328 - protein, 15 aa, Staphylococcus aureus
SGVMEGNVITAEEVK

SEQ ID NO: 329 - protein, 19 aa, Staphylococcus aureus
IIEQIGTYNPTSANAPEIK

SEQ ID NO: 330 - protein, 9 aa, Staphylococcus aureus
SQSVLVFAK

SEQ ID NO: 331 - protein, 10 aa, Staphylococcus aureus
VLELVGVGYR

SEQ ID NO: 332 - protein, 10 aa, Staphylococcus aureus
MAVEEIFNVK

SEQ ID NO: 333 - protein, 12 aa, Staphylococcus aureus
NYAVEATPGNLK

SEQ ID NO: 334 - protein, 7 aa, Staphylococcus aureus
AGIDINR

SEQ ID NO: 335 - protein, 10 aa, Staphylococcus aureus
VVVEGVNIMK

SEQ ID NO: 336 - protein, 12 aa, Staphylococcus aureus
ILEEANVSADTR

SEQ ID NO: 337 - protein, 8 aa, Staphylococcus aureus
VDEALALK

SEQ ID NO: 338 - protein, 14 aa, Staphylococcus coagulase negative
AVLELAGITDILSK

SEQ ID NO: 339 - protein, 11 aa, Staphylococcus coagulase negative
VIFLDTTTDFK

SEQ ID NO: 340 - protein, 10 aa, Staphylococcus coagulase negative
MFAIIETGGK

SEQ ID NO: 341 - protein, 8 aa, Staphylococcus coagulase negative
IDEELALK

SEQ ID NO: 342 - protein, 8 aa, Staphylococcus coagulase negative
SLLQPLPK

SEQ ID NO: 343 - protein, 11 aa, Staphylococcus pettenkorferi
VIFLDTTTNYK

SEQ ID NO: 344 - protein, 11 aa, Staphylococcus saprophyticus
ALINNMIQGVK

SEQ ID NO: 345 - protein, 10 aa, Staphylococcus saprophyticus
ILYSAFDLVK

SEQ ID NO: 346 - protein, 12 aa, Stenotrophomonas maltophilia
AYAFEDAINILK

SEQ ID NO: 347 - protein, 15 aa, Stenotrophomonas maltophilia
DFSEDLVHQVVVAYR

SEQ ID NO: 348 - protein, 12 aa, Stenotrophomonas maltophilia
DTAEVLLYALDK

SEQ ID NO: 349 - protein, 9 aa, Streptococcus agalactiae
TQLESETTR

SEQ ID NO: 350 - protein, 8 aa, Streptococcus agalactiae
GQVPGVTK

SEQ ID NO: 351 - protein, 12 aa, Streptococcus agalactiae
EGASEAEANEIK

SEQ ID NO: 352 - protein, 9 aa, Streptococcus agalactiae
SMVALEAGK

SEQ ID NO: 353 - protein, 11 aa, Streptococcus agalactiae
GLTVEQDTNLR

SEQ ID NO: 354 - protein, 12 aa, Streptococcus agalactiae
FTSVEEINALAK

SEQ ID NO: 355 - protein, 11 aa, Streptococcus agalactiae
LEAAGASVTLK

SEQ ID NO: 356 - protein, 12 aa, Streptococcus agalactiae
DVLSAGQEVTVK

SEQ ID NO: 357 - protein, 11 aa, Streptococcus agalactiae
AIVDNAPSVIK

SEQ ID NO: 358 - protein, 12 aa, Streptococcus agalactiae
GTHIYPGANVGR

SEQ ID NO: 359 - protein, 7 aa, Streptococcus agalactiae
SDIPEFR

SEQ ID NO: 360 - protein, 10 aa, Streptococcus bovis
FDETTGDYSR

SEQ ID NO: 361 - protein, 8 aa, Streptococcus bovis
AEDVAALR

SEQ ID NO: 362 - protein, 16 aa, Streptococcus bovis
TAEFANVLSALNVDSK

SEQ ID NO: 363 - protein, 15 aa, Streptococcus bovis
GTAASIVYDAFEQIK

SEQ ID NO: 364 - protein, 7 aa, Streptococcus bovis
LTAPSVK

SEQ ID NO: 365 - protein, 9 aa, Streptococcus bovis
TVAALGLGK

SEQ ID NO: 366 - protein, 13 aa, Streptococcus bovis
FIQTELADASVSR

SEQ ID NO: 367 - protein, 8 aa, Streptococcus bovis
EVVPAENR

SEQ ID NO: 368 - protein, 14 aa, Streptococcus bovis
NEIASENFDEATEK

SEQ ID NO: 369 - protein, 10 aa, Streptococcus bovis
IAGVDIPNEK

SEQ ID NO: 370 - protein, 8 aa, Streptococccus common
DFHGVPTK

SEQ ID NO: 371 - protein, 13 aa, Streptococccus common
NGIHVIDLQQTVK

SEQ ID NO: 372 - protein, 6 aa, Streptococcus common
VLVFAR

SEQ ID NO: 373 - protein, 12 aa, Streptococcus common
SLGSNTPINIVR

SEQ ID NO: 374 - protein, 7 aa, Streptococcus common
MIEGTAR

SEQ ID NO: 375 - protein, 7 aa, Streptococcus common
INVADSR

SEQ ID NO: 376 - protein, 10 aa, Streptococcus common
AIITLTADSK

SEQ ID NO: 377 - protein, 7 aa, Streptococcus common
LGLATTR

SEQ ID NO: 378 - protein, 10 aa, Streptococcus dysgalactiae
VEAGQVISVR

SEQ ID NO: 379 - protein, 7 aa, Streptococcus dysgalactiae
VINDFAK

SEQ ID NO: 380 - protein, 10 aa, Streptococcus dysgalactiae
VVVEGVGMIK

SEQ ID NO: 381 - protein, 7 aa, Streptococcus dysgalactiae
VLEWLAK

SEQ ID NO: 382 - protein, 15 aa, Streptococcus other streptococcus
AADGQTVTGGSILYR

SEQ ID NO: 383 - protein, 11 aa, Streptococcus other streptococcus
QAVEAAFEGVK

SEQ ID NO: 384 - protein, 10 aa, Streptococcus other streptococcus
IVSGPEADIK

SEQ ID NO: 385 - protein, 13 aa, Streptococcus other streptococcus
FVAVDSLSFTAPK

SEQ ID NO: 386 - protein, 11 aa, Streptococcus other streptococcus
IQIFEGVVIAR

SEQ ID NO: 387 - protein, 10 aa, Streptococcus pneumoniae
AAGDYEGLSK

SEQ ID NO: 388 - protein, 9 aa, Streptococcus pneumoniae
FVGQEFDTK

SEQ ID NO: 389 - protein, 9 aa, Streptococcus pyogenes
SAEAAIIAK

SEQ ID NO: 390 - protein, 10 aa, Streptococcus pyogenes
VDPGQVISVR

SEQ ID NO: 391 - protein, 7 aa, Streptococcus pyogenes
VINDFTK

SEQ ID NO: 392 - protein, 10 aa, Streptococcus pyogenes
VIVEGVGMIK

SEQ ID NO: 393 - protein, 14 aa, Citrobacter amalonaticus
AADMTGADIEAMTR

SEQ ID NO: 394 - protein, 11 aa, Citrobacter amalonaticus
LADVLAAANAR

SEQ ID NO: 395 - protein, 12 aa, Citrobacter amalonaticus
INALETVTITSK

SEQ ID NO: 396 - protein, 9 aa, Bacteroides fragili
MEVVNALGR

SEQ ID NO: 397 - protein, 10 aa, Bacteroides fragili
YLTPPSVDVK

SEQ ID NO: 398 - protein, 14 aa, Citrobacter braakii
YTAAITGAEGTIHR

SEQ ID NO: 399 - protein, 20 aa, Citrobacter braakii
TLNDAVAVNHADNALTFGPR

SEQ ID NO: 400 - protein, 14 aa, Citrobacter braakii
AGDQVQSGVDAAIK

SEQ ID NO: 401 - protein, 14 aa, Klebsiella pneumoniae
YTGAITAAAGTIHR

SEQ ID NO: 402 - protein, 9 aa, Proteus mirabilis
AAFAALVEK

SEQ ID NO: 403 - protein, 8 aa, Proteus vulgari
SIVVAIDR

SEQ ID NO: 404 - protein, 12 aa, Proteus vulgari
INALGSVTISSK

SEQ ID NO: 405 - protein, 13 aa, Providencia rettgeri
AANVVGIQIEYAK

SEQ ID NO: 406 - protein, 14 aa, Providencia rettgeri
AGDQIQSGVDSAIK

SEQ ID NO: 407 - protein, 11 aa, Providencia rettgeri
DMVESAPATIK

SEQ ID NO: 408 - protein, 12 aa, Providencia rettgeri
AAAFEGELIQAK

SEQ ID NO: 409 - protein, 18 aa, Providencia rettgeri
LSDFAAVEGDVIDLNALK

SEQ ID NO: 410 - protein, 10 aa, Staphylococcus capitis
SLELVGVGYR

SEQ ID NO: 411 - protein, 11 aa, Staphylococcus capitis
YNSEVTENLVK

SEQ ID NO: 412 - protein, 15 aa, Staphylococcus capitis
TGVMEGSVISAEEVK

SEQ ID NO: 413 - protein, 15 aa, Staphylococcus capitis
SGAEVSGPIPLPTEK

SEQ ID NO: 414 - protein, 8 aa, Staphylococcus capra
ELVDNAPK

SEQ ID NO: 415 - protein, 13 aa, Staphylococcus epidermidis
APGSVGMASDASK

SEQ ID NO: 416 - protein, 18 aa, Staphylococcus epidermidis
HIGSPNEVLEPGQQVNVK

SEQ ID NO: 417 - protein, 10 aa, Staphylococcus epidermidis
ILGIDEDNER

SEQ ID NO: 418 - protein, 9 aa, Staphylococcus hominis
YYSVEEAIK

SEQ ID NO: 419 - protein, 14 aa, Staphylococcus hominis
ILFEIAGVSEDVAR

SEQ ID NO: 420 - protein, 10 aa, Staphylococcus lugdunensis
ILGVDEDNER

SEQ ID NO: 421 - protein, 11 aa, Staphylococcus lugdunensis
VPAVVYGYSTK

SEQ ID NO: 422 - protein, 11 aa, Proteus vulgaris
LAETLAAAEAR

SEQ ID NO: 423 - protein, 11 aa, Proteus vulgaris
LYLTAAATAVR

SEQ ID NO: 424 - protein, 8 aa, Sus scrofa
NKPGVYTK

SEQ ID NO: 425 - protein, 8 aa, sus scrofa
VATVSLPR

SEQ ID NO: 426 - protein, 20 aa, Sus scrofa
LGEHNIDVLEGNEQFINAAK

SEQ ID NO: 427 - protein, 20 aa, Sus scrofa
IITHPNFNGNTLDNDIMLIK

SEQ ID NO: 428 - protein, 231 aa, Sus scrofa
FPTDDDDKIVGGYTCAANSIPYQVSLNSGSHFCGGSLINSQWVVSAAHCYKSRIQVRLGEHNIDVLEGNEQFINAAKIITHPNFNGNTLDNDIMLIKLSSPATLNSRVATVSLPRSCAAAGTECLISGWGNTKSSGSSYPSLLQCLKAPVLSDSSCKSSYPGQITGNMICVGFLEGGKDSCQGDSGGPVVCNGQLQGIVSWGYGCAQKNKPGVYTKVCNYVNWIQQTIAAN